(19) United States
(12) Patent Application Publication
(10) Pub. No.: US 2008/0044356 A1
(43) Pub. Date: Feb. 21, 2008

(54) Assemblies of Oligomeric Amyloid Beta Protein and Uses Thereof

(75) Inventors: Sylvain E. Lesne, Minneapolis, MN (US); Karen H. Ashe, North Oaks, MN (US); James P. Cleary, River Falls, WI (US)

Correspondence Address: MUETING, RAASCH & GEBHARDT, P.A., P.O. BOX 581415, MINNEAPOLIS, MN, 55458, US

(73) Assignee: Regents of the University of Minnesota, St. Paul, MN (US)

(21) Appl. No.: 11/665,670

(22) PCT Filed: Oct. 21, 2005
(86) PCT No.: PCT/US05/37828

§ 371 Date: Oct. 1, 2007

Related U.S. Application Data
(60) Provisional application No. 60/621,547, filed on Oct. 22, 2004.
(60) Provisional application No. 60/666,250, filed on Mar. 29, 2005.
(60) Provisional application No. 60/695,025, filed on Jun. 29, 2005.

(51) Int. Cl. A61K 39/395; A01K 67/00; A61K 49/00; C07K 16/18; G01N 33/53; C12P 1/00; C07K 14/00; A61K 39/00

(52) U.S. Cl. 424/9.2; 424/130.1; 424/184.1; 435/41; 435/7.1; 530/324; 530/387.1; 800/12

(57) ABSTRACT

The present invention provides assemblies of oligomeric amyloid beta protein and uses thereof.

### CONTINUING APPLICATION DATA

This application claims the benefit of U.S. Provisional Application Ser. No. 60/621,547, filed Oct. 22, 2004, U.S. Provisional Application Ser. No. 60/666,250, filed Mar. 29, 2005, and U.S. Provisional Application Ser. No. 60/695,025, filed Jun. 29, 2005, each of which is incorporated by reference herein.

### GOVERNMENT FUNDING

The present invention was made with government support under Grant No. R01-NS33249, awarded by the National Institutes of Health. The Government may have certain rights in this invention.

### BACKGROUND

The amyloid-β protein (Aβ) is implicated in the pathogenesis of Alzheimer's disease (AD). Functional imaging and neuropathological data support the fact that brain dysfunction in AD precedes neuron loss, leading to the prediction that specific forms of Aβ could disrupt memory before there is significant structural brain damage. The Aβ peptides are the major amyloid protein deposited in AD brains and both natural and synthetic forms have devastating effects on the viability and function of neurons. See, for example, Yankner et al., Science 250, 279-82 (1990); Pike et al., Brain Res 563, 311-4 (1991); Pike et al., J Neurosci 13, 1676-87 (1993); Lambert et al., Proc Natl Acad Sci USA 95, 6448-53 (1998); Walsh et al., Nature 416, 535-9 (2002); and Kayed et al., Science 300, 486-9 (2003). However, the identification and isolation of endogenous Aβ species causing brain dysfunction in AD has remained elusive because of the difficulty of correlating memory loss with distinct isolates of Aβ from subcellular compartments of the brain. There has been a long-standing debate as to whether the fibrillar, insoluble Aβ that is deposited in amyloid plaques disrupts memory in AD. The early studies showed an inverse relationship between amyloid load and cognitive function (Blessed et al., Br J Psychiatry 114, 797-811 (1968)). Nevertheless, the hypothesis that fibrillar Aβ is the cause of memory deficits has been undermined by the observation that some individuals with very high plaque loads have normal cognitive function (Crystal et al., Neurology 38, 1682-7 (1988); Arriagada et al., Neurology 42, 1681-8 (1992); Giannakopoulos et al., Arch Neurol 52, 1150-9 (1995); Lue et al., J Neuropathol Exp Neurol 55, 1083-8 (1996); and Davis et al., J Neuropathol Exp Neurol 58, 376-88 (1999)). In addition, several groups have recently demonstrated correlations between memory loss and soluble, non-fibrillar Aβ (Kuo et al., J Biol Chem 271, 4077-81 (1996); McLean et al., Ann Neurol 46, 860-6 (1999); and Lue et al., Am J Pathol 155, 853-62 (1999)). Yet, the precise form of the soluble Aβ responsible for the deficits in AD has not been established.

### SUMMARY OF THE INVENTION

The present invention includes isolated, soluble amyloid-β protein (Aβ) assemblies having more than one detergent stable oligomer of amyloid-β proteins. In some embodiments, the amyloid-β protein assembly disrupts cognitive function. In some embodiments, the amyloid-β protein assembly has a molecular weight of about 40 kilodaltons (kDa) as measured by SDS polyacrylamide gel electrophoresis. In some embodiments, the amyloid-β protein assembly has a molecular weight of about 56 kDa as measured by SDS polyacrylamide gel electrophoresis. In some embodiments, the amyloid-β protein assembly includes detergent stable dimers of amyloid-β protein. In some embodiments, the amyloid-β protein assembly includes detergent stable trimers of amyloid-β protein. In some embodiments, the amyloid-β protein assembly includes detergent stable tetramers of amyloid-β protein. In some embodiments, the amyloid-β protein assembly includes detergent stable hexamers of amyloid-β protein.

The present invention includes isolated, soluble amyloid-β protein assemblies having a dodecamer of amyloid-β proteins. In some embodiments, the dodecamer of amyloid-β proteins includes six detergent stable dimers of amyloid-O protein. In some embodiments, the dodecamer of amyloid-β proteins includes four detergent stable trimers of amyloid-β protein. In some embodiments, the dodecamer of amyloid-β proteins includes three detergent stable tetramers of amyloid-β protein. In some embodiments, the dodecamer of amyloid-β proteins includes two detergent stable hexamers of amyloid-β protein. In some embodiments, the dodecamer of amyloid-β proteins has a molecular weight of about 56 kDa as measured by SDS polyacrylamide gel electrophoresis.

The present invention includes isolated, soluble amyloid-β protein assemblies having more than one detergent stable trimer of amyloid-β proteins. In some embodiments, the amyloid-β protein assemblies include three detergent stable amyloid-β protein trimers. In some embodiments, the amyloid-β protein assemblies include four detergent stable amyloid-β protein trimers. In some embodiments, the amyloid-β protein assemblies have a molecular weight of about 40 kDa as measured by SDS polyacrylamide gel electrophoresis. In some embodiments, the amyloid-β protein assemblies have a molecular weight of about 56 kDa as measured by SDS polyacrylamide gel electrophoresis.

The present invention includes isolated, soluble amyloid-β protein assemblies having more than one detergent stable tetramer of amyloid-β proteins. In some embodiments, the amyloid-β protein assemblies include three detergent stable amyloid-β protein tetramers. In some embodiments, the amyloid-β protein assemblies have a molecular weight of about 56 kDa as measured by SDS polyacrylamide gel electrophoresis.

The isolated, soluble amyloid-β protein assemblies of the present invention may disrupt cognitive function.

The present invention includes compositions including isolated, soluble amyloid-β protein assemblies.

The present invention includes vaccines including isolated, soluble amyloid-β protein assemblies.

The present invention includes antibodies that bind to amyloid-β protein assemblies. In some embodiments, antibodies that bind to amyloid-β protein assemblies do not bind to monomeric amyloid-β protein. In some embodiments, antibodies that bind to amyloid-β protein assemblies do not bind to dimeric amyloid-β protein. In some embodiments, antibodies that bind to amyloid-β protein assemblies do not bind to trimeric amyloid-β protein. In some embodiments, antibodies that bind to amyloid-β protein assemblies do not bind to tetrameric amyloid-β protein.

The present invention includes a method of disrupting memory of learned behavior in a mammal, the method including administering amyloid-β protein assemblies intracranially. In some embodiments of the method, the mammal may be a mouse, rat, dog, or non-human primate. In some embodiments of the method, the amyloid-β protein assembly includes four detergent stable amyloid-β protein trimers. In some embodiments of the method, the amyloid-β protein assembly includes three detergent stable amyloid-β protein tetramers. In some embodiments of the method, the mammal serves an animal model system for a cognitive deficit. In some embodiments of the method, the mammal serves an animal model system for Alzheimer's disease. In some embodiments of the method, the mammal demonstrates cognitive deficits consistent with presymptomatic or early Alzheimer's disease.

The present invention includes an animal model, the animal model including a mammal wherein an amyloid-β protein assembly has been administered intracranially. In some embodiments of the animal model, the mammal is a mouse, rat, dog, or non-human primate. In some embodiments of animal model, the mammal demonstrates disruption of complex learned behaviors. In some embodiments of animal model, the mammal demonstrates cognitive deficits consistent with presymptomatic or early Alzheimer's disease.

The present invention includes a method of screening for an agent effective for the treatment of a cognitive disorder, the method including administering a test agent to a first animal to which a soluble amyloid-β protein assembly having more than one detergent stable oligomer of amyloid-β proteins has been intracranially administered; measuring cognitive function of the first animal; comparing the cognitive function of the first animal to the cognitive function of a second animal to which a soluble amyloid-β protein (Aβ) assembly having more than one detergent stable oligomer of amyloid-β proteins has been intracranially administered, but no test agent has been administered; wherein an improvement in the cognitive function of the first animal compared to the cognitive function of the second animal indicates the test agent is an effective agent for the treatment of a cognitive disorder. In some embodiments of the method, the cognitive disorder is a mild cognitive impairment. In some embodiments of the method, the cognitive disorder is an age related memory decline or an age associated memory impairment. In some embodiments of the method, the cognitive disorder is Alzheimer's disease.

The present invention includes a method of treating a cognitive disorder in a subject, the method including administering to the subject an agent that inhibits the assembly of monomers of amyloid β protein into soluble amyloid-β protein assemblies having more than one detergent stable oligomer of amyloid-β proteins. In some embodiments of the method, the cognitive disorder is a mild cognitive impairment. In some embodiments of the method, the cognitive disorder is an age related memory decline or an age associated memory impairment. In some embodiments of the method, the cognitive disorder is Alzheimer's disease.

The present invention includes agents that inhibit the assembly of detergent-stable oligomers of amyloid β protein into soluble amyloid-β protein assemblies. The present invention includes agents that promote the clearance of soluble amyloid-β protein assemblies from neurological tissue.

The present invention includes a method of treating a cognitive disorder in a subject, the method including administering to the subject an agent that inhibits the assembly of detergent-stable oligomers of amyloid β protein into a soluble amyloid-β protein assembly including more the one detergent stable oligomer of amyloid-β proteins. In some embodiments of the method, the cognitive disorder is a mild cognitive impairment. In some embodiments of the method, the cognitive disorder is an age related memory decline or an age associated memory impairment. In some embodiments of the method, the cognitive disorder is Alzheimer's disease.

The present invention includes a method of treating a cognitive disorder in a subject, the method including administering to the subject an agent that promotes the clearance from neurological tissue of a soluble amyloid-β protein assembly including more than one detergent-stable oligomer of amyloid-β proteins. In some embodiments of the method, the cognitive disorder is a mild cognitive impairment. In some embodiments of the method, the cognitive disorder is an age related memory decline or an age associated memory impairment. In some embodiments of the method, the cognitive disorder is Alzheimer's disease.

The present invention includes a method of detecting a cognitive disorder in a subject, the method including detecting in a fluid or tissue sample taken from the subject soluble amyloid-β protein assemblies including more than one detergent stable oligomer of amyloid-β proteins. In some embodiments of the method, the cognitive disorder is a mild cognitive impairment. In some embodiments of the method, the cognitive disorder is an age related memory decline or an age associated memory impairment. In some embodiments of the method, the cognitive disorder is Alzheimer's disease.

The present invention includes a method of detecting a presymptomatic cognitive disorder in a subject, the method including detecting in a fluid or tissue sample taken from the subject soluble amyloid-β protein assemblies including more than one detergent stable oligomer of amyloid-β proteins. In some embodiments of the method, the cognitive disorder is a mild cognitive impairment. In some embodiments of the method, the cognitive disorder is an age related memory decline or an age associated memory impairment. In some embodiments of the method, the cognitive disorder is Alzheimer's disease.

The present invention includes a method for assaying the effects of soluble oligomers of the amyloid β protein on cognitive function, the method including administering a soluble amyloid-β protein assembly comprising more than one detergent-stable oligomer of amyloid-β proteins intracranially into an animal and measuring cognitive function to determine the disruption of cognitive behavior. In some embodiments of the method, the disruption of cognitive behavior of the animal is compared to the long-term disruption of cognitive behavior of another animal treated in the same fashion except saline rather a soluble amyloid-β protein assembly comprising one or more one detergent-stable oligomer of amyloid-β proteins is administered intracranially. In some embodiments of the method, the cognitive disorder is a mild cognitive impairment. In some embodiments of the method, the cognitive disorder is an age related memory decline or an age associated memory impairment. In some embodiments of the method, the cognitive disorder is Alzheimer's disease.

The present invention includes methods of isolating soluble amyloid-β protein assemblies including more than one detergent-stable oligomer of amyloid-β proteins, the method including homogenizing neuronal tissue in a lysis buffer; size fractionating amyloid-β protein (Aβ) assemblies; and isolating an amyloid-β protein (Aβ) assembly of a desired size.

Unless otherwise specified, “a,” “an,” “the,” and “at least one” are used interchangeably and mean one or more than one.

### BRIEF DESCRIPTION OF THE FIGURES

FIGS. 1A-1C. Temporal patterns of memory decline and soluble, extracellular Aβ oligomers in Tg2576 mice. In FIG. 1A spatial memory in mice from 4 to 17 months shows a progressive but irregular decline with periods of stability. Tg2576^{+/-} (filled circles, ●), Tg2576^{-/-} (open circles, ∘). ANOVA, p<0.01, followed by t test, *p<0.01. In FIG. 1B a temporal analysis of spatial memory shows three stages of performance. Tg2576^{+/-} (filled bars), Tg2576^{-/-} (open bars). ANOVA, *p<0.01, **p<0.001. FIG. 1C is an identification of Aβ oligomers in soluble, extracellular-enriched extracts of proteins from brains of 5-, 6-, and 7-month mice, assessed by western blot (WB) with or without immunoprecipitation (IP). The intensity of the 40 kDa band co-migrating with nonamers was 33.92±12.5% (n=4) of the intensity of the 56 kDa band corresponding to dodecamers.

FIGS. 2A-2H. Water maze performance correlates inversely with Aβ nonamer and dodecamer levels. A lack of significant correlations between spatial memory and monomeric (FIG. 2A and FIG. 2D), trimeric (FIG. 2B and FIG. 2E), or hexameric (FIG. 2C and FIG. 2F) soluble Aβ species was detected in the extracellular-enriched fractions of 5-month (open circles, ∘) and 6-month (filled circles, ●) Tg2576+/− mice. Nonameric (FIG. 2G) and dodecameric (FIG. 2H) Aβ levels correlate inversely with spatial memory at 6 months (ANOVA).

FIGS. 3A-3C. Aβ*56 disrupts cognitive function. In FIG. 3A soluble proteins in extracellular-enriched extracts from Tg2576^{+/-} or Tg2576^{-/-} mice were fractionated by SEC to generate fractions with (19^{+/-}) or without (17^{+/-} and 19^{-/-}) the 56 kDa Aβ species. Bands at 75 and 150 kDa (arrows) present in non-Tg littermates and IP19^{+/-} were considered to be non-specific (ns). FIG. 3B presents Perseveration Errors, presented as a percentage of baseline rates (±SEM), in the ALCR paradigm two hours after rats received injections of the indicated fractions into the lateral ventricles. FIG. 3C presents Switching Errors, presented as a percentage of baseline rates (±SEM), in the ALCR paradigm two hours after rats received injections of the indicated fractions into the lateral ventricles. Wilcoxon matched-pairs signed-ranks test, *p<0.05.

FIGS. 4A-4E. Elimination of Aβ*56 coincides with an interlude of normal spatial memory. FIG. 4A shows Aβ*56 levels decline in Tg2576^{+/-} mice between 12.0-12.4 months of age. FIG. 4B shows levels of Aβ*56 between 10.7-13.0 months of age. Values represent band intensities (mean ±SD) relative to the intensities observed at 10.7 months. ANOVA, p<0.02, followed by t test, *p<0.01, n=4 animals per age group. In FIG. 4C the dip in Aβ*56 levels coincides with a transient recovery of spatial memory (target quadrant occupancy±SEM in Day 5 probe trial). ANOVA, p<0.01, followed by t test, *p<0.01, *p<0.001, and by Fisher's protected least significant difference (PLSD) test, ‡p<0.001, #p<0.05. Tg2576^{+/-} (filled bars), Tg2576^{-/-} (open bars). FIG. 4D demonstrates that the kinetics of the rate of change in Aβ(x-42) levels (SDS-soluble Aβ(x-42) [open circles, ∘] and SDS-insoluble Aβ(x-42) [filled squares, ▪]) in Tg2576^{+/-} mice. Abbreviations: FA=SDS-insoluble/formic acid-soluble; Δt=time interval. FIG. 4E presents a hypothetical dynamic relationship between soluble and insoluble pools of Aβ.

FIGS. 5A-5C. Fidelity of the technique for measuring soluble, extracellular Aβ oligomers in vivo. FIG. 5A illustrates the procedures used to extract Aβ. The forebrain is subjected to a four step extraction protocol generating four fractions (extracellular-enriched soluble (EC), intracellular-enriched soluble (IC), membrane-enriched (MB), and insoluble (Insol)). FIG. 5B presents SDSPAGE analyses of various protein markers in the four collected fractions (extracellular-enriched soluble (EC), intracellular-enriched soluble (IC), membrane-enriched (MB), and insoluble (Insol)). Partial characterization and validation of the fractionation procedure was achieved using the NMDA receptor subunit NR2 as a marker for membrane-enriched proteins, APP as a marker for both soluble (sAPPα) and membrane-enriched (full-length APP=fl-APP) proteins, and the extracellular serine protease tissue-type plasminogen activator t-PA, as a marker for soluble proteins. Other protein markers used were microtubule-associated proteins MAP-2 and tau (cytoskeleton), the protein kinases, ERKs and JNK (cytosol), and flotillin-2 (lipid rafts). FIG. 5C demonstrates a validation of the use of immunoprecipitation to study Aβ species quantitatively. To ensure that immunoprecipitated Aβ species were a faithful index of brain Aβ levels, two sequential immunoprecipitations (IP1 and IP2) with either 6E10 or 4G8 monoclonal antibodies were performed and revealed negligible amounts (1±0.64%, n=4) of 6E10 immunoreactive material in western blot after the first immunoprecipitation in extracts from the oldest animals (25 months).

FIGS. 6A-6D. Biochemical and structural properties of Aβ assemblies in Tg2576 mice. FIG. 6A shows soluble, extracellular-enriched Aβ species purified using affinity columns packed with 200 μg of 6E10 or 4G8 antibodies. Captured proteins were eluted in acidic buffer (pH 3), fractionated by SDS-PAGE, and WB were revealed with 6E10. FIG. 6B shows multimers are resistant to the strong chaotropic agent, 8M urea. Soluble, extracellular-enriched extracts from 12- to 20-month Tg2576+/− brains were loaded onto 8M urea containing SDS-PAGE gels, electrotransfered, and probed with 6E10. The presence of urea did not alter electrophoretic migration patterns, indicating that Aβ oligomers are probably not associated with large globular proteins. FIG. 6C shows soluble HMW Aβ oligomers are not resistant to treatment with ≧15% hexafluoroisopropanol (HFIP). Monomeric Aβ levels increased with rising HFIP concentrations. Trimers were exceptionally resistant to HFIP. FIG. 6D is an evaluation of Aβ multimers with the anti-oligomer antibody, A11.

FIGS. 7A-7D. Characterization of native Aβ oligomer size and expression levels in Tg2576 mice. FIG. 7A is a SDS-PAGE analysis of soluble brain extracts fractionated by SDS-free-size-exclusion chromatography (SEC) showed that all Aβ oligomers migrated at expected molecular weights using globular protein standards. Bands revealed at 75 and 150 kDa are non-specific bands which are also present in blots of extracts from non-transgenic mice. FIG. 7B shows soluble, extracellular-enriched Aβ species assessed by western blot using 6E10 in mice between 9 and 25 months of age. FIG. 7C represents a semi-quantification of Aβ species levels (relative to β-tubulin levels) expressed as percentage of respective averaged signals observed in 9-month-old animals (n=6/age group) for 1-mer, 3-mer, 4-mer, 6-mer, and 9-mer. FIG. 7D 7C represents a semi-quantification of Aβ species levels (relative to β-tubulin levels) expressed as percentage of respective averaged signals observed in 9-month-old animals (n=6/age group) for 12-mers. ANOVA, followed by t test, *p<0.01, # p<0.05.

FIGS. 8A and 8B. Absence of correlation between Aβ oligomer levels and swimming speed or path length during the cued (or visible) phase of water maze testing. FIG. 8A shows the relationship between swimming speed and Aβ levels in 5-month-old Tg2576+/− mice. FIG. 8B shows the relationship between swimming speed and Aβ levels in 6-month-old Tg2576+/− mice. ANOVA p values are displayed in graphs alongside r2 values.

FIGS. 9A-9C. No change in levels of intracellular Aβ levels and in CTFs in 5- and 6-month Tg2576 mice. In FIG. 9A soluble, intracellular-enriched As species in 5-, 6- and 7-month mice was evaluated by western blot (WB) using 6E10. FIG. 9A shows levels of sAPPα. FIG. 9B demonstrates compartmentalization of APP-CTFs in Tg2576 mouse brain. CTF-βs were immunoprecipitated (IP'd) from soluble or membrane extracts with 6E10 and identified in WB with APPCter-C17. No immunoreactive bands were detected in the soluble fraction, but a doublet of bands around approximately 12 kDa was present in the membrane-enriched fraction. As a control, both CTF-β and CTF-α were detected with APPCter-C17. In FIG. 9C IPs of CTFs using APPCter-C17 confirmed no overall change in CTFs between 5 to 7 months of age. Full-length APP also captured by the antiserum is displayed on top and shows no variation in levels.

FIGS. 10A-10E. Trimers are the predominant oligomeric Aβ species secreted from Tg2576 cultured primary brain cells. FIG. 10A shows levels of naturally secreted Aβ species in the conditioned media (CM) of 7- or 14-DIV (days in vitro)-old neurons evaluated by IP followed by WB with 6E10 antibodies. FIG. 10B shows cortical astrocytes modulate the levels of neuron-derived Aβ species in the CM. FIG. 10C shows boiling the membranes enhances the detection of monomeric AD with 6E10, which constitutes the major Aβ species secreted in the CM. However, boiling did not significantly enhance the ability to detect the oligomeric Aβ species. FIG. 10D shows intracellular protein preparations from Tg2576+/− primary neurons devoid of APP-CTFs were IP'd with 6E10, revealing trimers but not tetramers. FIG. 10E shows membrane-associated APP-derived molecules in Tg2576+/− primary neurons. IPs using 6E10 captures CTF-βs and Aβ monomers. The blot was denatured and re-probed with APPCter-C17 to confirm the nature of the approximately 13 kDa bands, revealing both phosphorylated (pCTF) and nonphosphorylated CTF-βs.

FIG. 11. Overall response rates. Response rates of rats were compared at baseline and after receiving fractions from Tg Pos Lane 19 containing Aβ*56 from aTg2576 transgene positive mouse (corresponding Tg Neg Lane 19 from a transgene negative mouse), and Tg Control Lane 17 without Aβ*56 from a transgene positive mouse.

FIG. 12. Running response rates of rats compared at baseline and after receiving fractions containing Aβ*56 from a Tg2576 transgene positive mouse and Tg2576 Control Ln 17 without Aβ*56 from a transgene positive mouse.

FIG. 13. Post reinforcement pause as a function of ratio size. Post reinforcement pause was compared as a function of ratio size in rats at baseline and after receiving fractions containing Aβ*56 from a Tg2576 transgene positive mouse and Tg2576 Control Ln 17 without Aβ*56 from a transgene positive mouse

FIG. 14. Extracellular-enriched Tg2576 extracts from the B6SJL and 129FVBF1 strains of mice.

FIG. 15. Methods for screening candidate monoclonals for antibodies that specifically detect Aβ*56.

FIGS. 16A-16C. Human-derived AO*56 physically binds NMDA receptors. FIG. 16A demonstrates that Aβ*56 coimmunoprecipitates with NR1 NMDA receptor subunits in brain tissue from Alzheimer (AD) patients but not from control subjects with no cognitive impairment (NCI), or extracts containing no brain proteins (NP). FIG. 16B demonstrates that Aβ*56 co-immunoprecipitates with NR2A, but much less readily with NR2B, NMDA receptor subunits in brain tissue from subjects with AD but not from control subjects (NCI). FIG. 16C demonstrates that Aβ*56 does not co-immunoprecipitate with a7 nicotinic acetylcholine receptors (α7nAChR). Panels below each blot confirm the ability of the various receptor antibodies to immunoprecipitate the respective receptors or receptor subunits.

### DETAILED DESCRIPTION OF ILLUSTRATIVE EMBODIMENTS OF THE INVENTION

The present invention shows, for the first time, that cognitive deficits occur as a result of the accumulation of soluble assemblies of oligomers of amyloid-β proteins. The soluble amyloid-β protein assemblies of the present invention are made up of one or more detergent-stable oligomers of amyloid-β protein. In some embodiments, the soluble amyloid-β protein assemblies are made up of more than one detergent-stable oligomer of amyloid-β protein. The soluble amyloid-β protein (Aβ) assemblies of the present invention may also be referred to herein as Aβ* assemblies, Aβ* molecules, Aβ star assemblies, Aβ star molecules, A-beta* assemblies, A-beta* molecules, A-beta star assemblies, A-beta star molecules, Aβ*, Aβ*56, or Aβ star 56.

The soluble amyloid-β protein assemblies of the present invention may be isolated. The soluble amyloid-β protein assemblies of the present invention may be purified. The soluble amyloid-β protein assemblies of the present invention may be isolated and purified. As used herein, the term “isolated” means that a polypeptide, oligomer of polypeptides, or assembly of oligomers is either removed from its natural environment or synthetically derived, for instance by recombinant techniques, or chemically or enzymatically synthesized. As used herein, the term “purified” means that a polypeptide, oligomer of polypeptides, or assembly of oligomers is essentially free from any other polynucleotides or polypeptides and associated cellular products or other impurities.

As used herein an “amyloid-beta protein,” also referred to as an “amyloid-beta polypeptide,” an “amyloid-beta peptide,” an amyloid-beta molecule,” an “amyloid-β protein,” an “amyloid-β polypeptide,” an “amyloid-β peptide,” and “amyloid-β molecule,” an “Aβ protein,” an “AD polypeptide,” an “Aβ peptide,” an “Aβ molecule,” “amyloid-beta,” “amyloid β,” or “Aβ,” is the major constituent of amyloid plaques in the brains of individuals afflicted with Alzheimer's disease, a polypeptide of 39 to 43 amino acid residue first identified by Glenner and Wong (see, for example, Glenner et al., (1984) Biochem Biophys. Res Conumun 120, 885-890; and Glenner and Wong (1984) Biochem Biophys Res Commun 122, 1131-1135) and Masters et al. (See Masters et al., (1985) Embo J 4, 2757-2764; and Masters et al., (1985) Proc Natl Acad Sci USA 82, 4245-4249). The gene for the amyloid precursor protein (APP) of the amyloid-beta protein has been cloned and sequenced (see, for example, Kang et al., (1987) Nature 325, 733; Tanzi et al., (1987) Science 235, 880-884; and Selkoe (1994) Annual Review of Neuroscience Vol, 17, 489-517). As used herein, an amyloid-beta protein may be any of the various known allelic variants and mutations of the amyloid-beta protein.

Amyloid beta peptide is generated from the beta-amyloid precursor protein (beta APP) in a two-step process. The first step involves cleavage of the extracellular, amino-terminal domain of beta APP. Protein cleavage is performed by an aspartyl protease termed beta-secretase (BACE). This enzyme is synthesized as a propeptide that must be modified to the mature and active form by the prohormone convertase, furin. Beta APP cleavage by the mature form of BACE results in the cellular secretion of a segment of beta APP and a membrane-bound remnant. This remnant is then processed by another protease termed gamma-secretase. Gamma-secretase cleaves an intra-membrane site in the carboxyl-terminal domain of beta APP, thus generating the amyloid beta peptide. Gamma-secretase is believed to be a multi-subunit complex containing presenilin-1 and 2 as central components. Found associated with the presenilins is the transmembrane glycoprotein nicastrin. Nicastrin has been found to bind to the carboxyl-terminus of betaAPP and helps to modulate the production of the amyloid beta peptide. Also found in the neurofibrillary lesions in Alzheimer's disease is the protein termed Tau. Tau is a neuronal microtubule-associated protein found predominantly on axons. The function of tau is to promote tubulin polymerization and stabilize microtubules. Tau, in its hyperphosphorylated form, is the major component of paired helical filaments (PHF), which is the building block of neurofibrillary lesions in Alzheimer's disease brain. See, for example, J. Neurosci. 18:1743-1752, 1998 and Neuron, 19:939-945, 1997.

As used herein, an amyloid-beta protein is a monomeric polypeptide, made up of one polypeptide chain. A monomeric polypeptide is also referred to herein as a “monomer.”

As used herein an oligomer of amyloid β, also referred to an oligomeric form of amyloid β, is a detergent-stable configuration of more than one amyloid-beta protein. An oligomer is not necessarily polymerized. An oligomer of amyloid β may be soluble. As used herein a “dimer” is a detergent-stable configuration of two amyloid-beta proteins. As used herein a “trimer” is a detergent-stable configuration of three amyloid-beta proteins. As used herein a “tetramer” is a detergent-stable configuration of four amyloid-beta proteins. As used herein a “pentamer” is a detergent-stable configuration of five amyloid-beta proteins. As used herein a “hexamer” is a detergent-stable configuration of six amyloid-beta proteins.

As used herein, an “assembly” is a configuration of one or more oligomers of Aβ proteins. In a preferred embodiment, an assembly is a configuration of more than one Aβ protein oligomers. An assembly of oligomers of Aβ proteins may be, for example, an assembly of two oligomers of Aβ proteins, three oligomers of Aβ proteins, four oligomers of Aβ proteins, five oligomers of Aβ proteins, six oligomers of Aβ proteins, or more oligomers of Aβ proteins. In some embodiments, an assembly of oligomers of Aβ proteins may be, for example, a nanomer of nine amyloid β proteins or a dodecamer of twelve amyloid β proteins. In some embodiments, an assembly of oligomers of Aβ proteins may be, for example, an assembly of more than one hexamer of amyloid β proteins, more than one pentamer of amyloid β proteins, more than one tetramer of amyloid β proteins, more than one trimer of amyloid β proteins, or more than one dimer of amyloid β proteins. In some embodiments, an assembly of oligomers of Aβ proteins may be, for example, an assembly of two hexamers of amyloid β proteins, three hexamers of amyloid β proteins, two tetramers of amyloid β proteins, three tetramers of amyloid β proteins, four tetramers of amyloid β proteins, two trimers amyloid β proteins, three trimers amyloid β proteins, four trimers of amyloid β proteins, five trimers amyloid β proteins, two dimers of amyloid β proteins, three dimers of amyloid β proteins, four dimers of amyloid β proteins, five dimers of amyloid P proteins, six dimers of amyloid β proteins, seven dimers of amyloid β proteins, or eight dimers of amyloid β proteins.

In some embodiments, amyloid-β protein assemblies may include detergent-stable dimers of amyloid-β protein. In some embodiments, amyloid-β protein assemblies may include detergent-stable trimers of amyloid-β protein. In some embodiments, amyloid-β protein assemblies may include detergent-stable tetramers of amyloid-β protein. In some embodiments, amyloid-β protein assemblies may include detergent-stable pentamers of amyloid-β protein. In some embodiments, amyloid-β protein assemblies may include detergent-stable hexamers of amyloid-β protein.

The present invention also includes isolated, soluble amyloid-β protein assemblies having one or more amyloid-β protein trimers. As used herein an “amyloid-β protein trimer” is a detergent-stable configuration of three A13 molecules. In some embodiments, a soluble amyloid-β protein assembly has more than one amyloid-β protein trimer. In some embodiments, the amyloid-β protein assembly includes three amyloid-β protein trimers. In some embodiments, the amyloid-β protein assembly is a nonamer of amyloid-β proteins. In some embodiments, an amyloid-β protein assembly has a molecular weight of about 40 kDa as measured by SDS polyacrylamide gel electrophoresis. In some embodiments, the amyloid-β protein assembly includes four amyloid-β protein trimers. In some embodiments, the amyloid-β protein assembly has a molecular weight of about 56 kDa as measured by SDS polyacrylamide gel electrophoresis.

In some embodiments, amyloid-β protein assemblies may be a dodecamer of amyloid-β proteins. Such dodecamers of amyloid-β proteins may be six dimers of amyloid-β protein, four trimers of amyloid-β protein, three tetramers of amyloid-β protein, or two hexamers of amyloid-β protein. In some embodiments, the dodecamer of amyloid-β proteins has a molecular weight of about 56 kDa as measured by SDS polyacrylamide gel electrophoresis.

As used herein, a detergent-stable, also referred to herein as “detergent stable,” configuration does not disassemble or disassociate into its component subunits in a detergent solution. Such a detergent solution may be, for example, a 1% solution Triton X-100 or a 2% solution of SDS. Thus, a detergent stable oligomer of amyloid-β protein does not disassociate into separate amyloid-O protein monomers in a detergent solution. Likewise, a detergent stable assembly of oligomers of amyloid-β protein does not disassociate into separate oligomers of amyloid-β proteins in a detergent solution.

The assemblies of amyloid β protein of the present invention are soluble. As used herein, the term “soluble” means remaining in aqueous solution. In some embodiments, soluble assemblies of amyloid β protein remain in the supernatant after centrifugation, including, for example, ultracentrifugation. Soluble assemblies of amyloid β protein may remain in solution in a wide range of solutions, including, but not limited to, water, in an isotonic solution, tissue culture medium, a buffered solution, a detergent buffer, an organic buffer, or a body fluid, including, for example, plasma or cerebrospinal fluid. Assemblies of amyloid P protein may remain in solution in a physiological buffer.

Assemblies of amyloid β protein may remain in solution in range of temperatures. For example, the assemblies of amyloid β protein may remain in solution at a temperature greater than 0° C. Assemblies of amyloid β protein may remain in solution, for example, at a temperature of at least about 4° C., at a temperature of at least about 10° C., at a temperature of at least about 15° C., at a temperature of at least about 25° C., at a temperature of at least about 37° C., at a temperature of at least about 42° C., at a temperature of at least about 50° C., at a temperature of at least about 55° C., at a temperature of at least about 60° C., at a temperature of at least about 70° C., at a temperature of at least about 75° C., at a temperature of at least about 80° C., at a temperature of at least about 85° C., at a temperature of at least about 90° C., at a temperature of at least about and/or at a temperature of at least about 95° C.

Assemblies of amyloid β protein may remain in solution, for example, at a temperature of less than about 4° C., at a temperature of less than about 10° C., at a temperature of less than about 15° C., at a temperature of less than about 25° C., at a temperature of less than about 37° C., at a temperature of less than about 42° C., at a temperature of less than about 50° C., at a temperature of less than about 55° C., at a temperature of less than about 60° C., at a temperature of less than about 70° C., at a temperature of less than about 75° C., at a temperature of less than about 80° C., at a temperature of less than about 85° C., at a temperature of less about 90° C., at a temperature of less than about 95° C., and/or at a temperature of less than about 100° C.

Assemblies of amyloid β protein may remain in solution, for example, at a temperature of about 4° C., at a temperature of about 10° C., at a temperature of about 15° C., at a temperature about 25° C., at a temperature of about 37° C., at a temperature of about 42° C., at a temperature of at about 50° C., at a temperature of about 55° C., at a temperature of about 60° C., at a temperature of about 70° C., at a temperature of about 75° C., at a temperature of at about 80° C., at a temperature of about 85° C., at a temperature of about 90° C., and/or at a temperature of about 95° C.

Assemblies of amyloid β protein may remain in solution in a range of any of the various temperatures discussed above.

In some embodiments, the oligomers or assemblies of amyloid-β protein are non-fibrillar. As used herein, a “non-fibrillar” protein, also referred to herein a “globular” protein, has little alpha helical or beta sheet structure. As used herein, a fibrillar protein has extensive alpha helix or beta sheet structure. The oligomers or assemblies of amyloid β protein may be preparations from which the fibrillar form of amyloid β is absent or has been removed. See, for example, U.S. Pat. No. 6,218,506 and Walsh et al., (2002) Nature 416, 535-539 for a more complete discussion of the non-fibrillar structure of amyloid beta.

Both assemblies of amyloid β protein and oligomers of amyloid β protein may be obtained from a wide variety of sources. Assemblies of amyloid β protein and oligomers of amyloid β protein may be obtained from natural sources; for example, from natural fluids, cells, or tissues, including, but not limited to, plasma, brain tissue, and cerebrospinal fluid. Assemblies of amyloid β protein and oligomers of amyloid β may be isolated from the culture medium of cells expressing endogenous or transfected amyloid β protein precursor genes. For example, assemblies amyloid β or oligomers of amyloid β protein may be obtained from the culture medium of Chinese hamster ovary (CHO) cells stably transfected to express amyloid β protein (Podlinsky et al., J. Biol. Chem., 1995, 270(16):9564-9570). Assemblies of amyloid β protein and oligomers of amyloid β protein may be synthetically produced. Assemblies of amyloid β protein and oligomers of amyloid β protein may be produced recombinantly

Amyloid-β protein assemblies of the present invention disrupt cognitive functioning, representative of a cognitive disorder. Such cognitive disorders include, but are not limited to, mild cognitive impairment, memory deficits, age related memory decline, age associated memory impairment, and Alzheimer's disease, including, but not limited to presymptomatic Alzheimer's disease and early Alzheimer's disease. Disruptions of cognitive function may be representative of any phase of a neurological disorder, including, but not limited to, a presymptomatic phase, a preclinical phase, or an early phase of a neurological disorder. The disruption of cognitive function may be representative of age-related memory decline or age-associated memory impairment (see Craik, F. I. in Handbook of the Psychology of Aging (eds. Birren, J. E. & Schall, K.) 384-420 (Van Nostrand-Reinhold, New York, 1977) and Morrison and H of, Science 1997, 278; 412-9).

Cognitive disruption may be assayed by any of a variety of methods. One means of assessing cognitive functioning is the Alternating Lever Cyclic Ratio (ALCR) test, which has proven to be sensitive for measuring cognitive function (O'Hare et al., Behav Pharmacol 1996, 7:742-753; and Richardson et al., Brain Res 2002, 954:1). Under ALCR, rats learn a complex sequence of lever-pressing requirements for food reinforcement in a two-lever experimental chamber. Rats must alternate between the two levers, switching to the other lever after pressing the first lever enough to get a food pellet. The number of presses required for each food reward proceeds from low (2 presses) to high (56 presses), incorporating intermediate values based on the quadratic function, x²−x. One cycle is an entire ascending and descending sequence of these response requirements (for example, 2, 6, 12, 20, 30, 42, 56, 56, 42, 30, 20, 12, 6, and 2 presses per food reward). Six such full cycles are presented during each session. Errors are scored when the subject perseveres on a lever after reward, that is, does not alternate (a perseveration error), or when a subject switches levers before completing the response requirement on that lever (a switching error).

Other procedures that may be used to assess cognitive functioning, include, but are not limited to, a delayed non-matching to place test, a morris water maze (commonly used to assess working memory in rats and mice), a delayed matching to sample test (an operant procedure for testing working memory), and a fixed-interval operant responding test (a sensitive procedure to assess non-specific cognitive effects, for example, when the type and anatomical location of the cognition being tested is unknown), a delayed conditioning procedure (representing a variety of operant or non-operant tests under which animals are exposed to stimuli paired with a reward or punishment and, after a delay, their ability to respond appropriately to the stimulus-reward combination is assessed), or a repeated acquisition procedure (an operant test, under which subjects are required to repeatedly learn a new stimulus sequence).

With the present invention, the accumulation of assemblies of oligomers of amyloid β is associated with in one or more neurological functional deficiencies. Such functional deficiencies may be transient or permanent. Such functional deficiencies may be observed in the absence of neuropathological damage. Such neuropathologies may include, for example, amyloid plaque formation, amyloid deposits, oxidative stress, astrogliosis, microgliosis, cytokine production, dystrophic neurons, formation of neurobifillary tangles, neurodegeneration, gross neuronal atrophy, neuronal loss, synaptic loss, and other manifestations of neuropathology.

Also included in the present invention are compositions including one or more of the soluble amyloid-β protein assemblies described herein. A composition may include one or more accessory ingredients including, but not limited to, diluents, buffers, binders, disintegrants, surface active agents, thickeners, lubricants, preservatives (including antioxidants), solvents, diluents, antibacterial and antifungal agents, absorption delaying agents, carrier solutions, suspensions, colloids, and the like. A composition may further include additional therapeutic agents. The preparation and use of such compositions is well known in the art.

A composition may be a pharmaceutical acceptable composition, meaning that the composition is not biologically or otherwise undesirable, i.e., the material may be administered to an individual without causing any undesirable biological effects or interacting in a deleterious manner with any of the other components of the pharmaceutical composition in which it is contained. Also included in the present invention are vaccines including one or more of the amyloid-β protein assemblies described herein. The preparation and use of such vaccines is well known in the art.

The present invention includes antibodies that bind to the amyloid-β protein assemblies described herein. In some embodiments, an antibody binds to an amyloid-β protein assembly and does not bind to monomeric amyloid-β protein. In some embodiments, an antibody binds to an amyloid-β protein assembly and does not bind to dimeric amyloid-β protein. In some embodiments, an antibody binds to an amyloid-β protein assembly and does not bind to trimeric amyloid-β protein. In some embodiments, an antibody binds to an amyloid-β protein assembly and does not bind to tetrameric amyloid-β protein. In some embodiments, an antibody binds to an amyloid-β protein assembly and does not bind to the amino-terminal region of the amyloid-β protein. In some embodiments, an antibody binds to an amyloid-β protein assembly and does not bind to the mid-region of the amyloid-β protein. In some embodiments, an antibody binds to an amyloid-β protein assembly and does not bind to the carboxyl-terminal region of the amyloid-β protein. Also included in the present invention are compositions including one or more of the antibodies as described herein.

As used herein, the terms “antibody” or “antibodies” includes polyclonal antibodies, affinity-purified polyclonal antibodies, monoclonal antibodies, and antigen-binding fragments thereof, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. The term “polyclonal antibody” refers to an antibody produced from more than a single clone of plasma cells; in contrast “monoclonal antibody” refers to an antibody produced from a single clone of plasma cells. Polyclonal antibodies may be obtained by immunizing a variety of warm-blooded animals such as horses, cows, goats, sheep, dogs, chickens, rabbits, mice, hamsters, guinea pigs and rats as well as transgenic animals such as transgenic sheep, cows, goats or pigs, with an immunogen. The resulting antibodies may be isolated from other proteins by using an affinity column having an Fc binding moiety, such as protein A, or the like. Monoclonal antibodies can be obtained by various techniques familiar to those skilled in the art. Briefly, spleen cells from an animal immunized with a desired antigen are immortalized, commonly by fusion with a myeloma cell [see, Kohler and Milstein (1976) Eur. J. Immunol. 6, 511-519; J. Goding (1986) In “Monoclonal Antibodies: Principles and Practice,” Academic Press, pp 59-103]. Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Isolated assemblies of oligomers of amyloid β protein, oligomers of amyloid β protein, or fragments thereof may serve as an antigen to immunize an animal to elicit an immune response. Immunization with antigen may be accomplished in the presence or absence of an adjuvant, e.g., Freund's adjuvant. Booster immunizations may be given at intervals, e.g., 2-8 weeks. Both polyclonal and monoclonal antibodies may be labeled with detectable label using methods known in the art. For example, fluorescent labels or peroxidase may be used as detectable labels. Various techniques useful in these arts are discussed, for example, in Harlow and Lane, (1988) “Antibodies: A Laboratory Manual,” Cold Spring Harbor, N.Y.

A therapeutically useful antibody may be derived from a “humanized” monoclonal antibody. Humanized monoclonal antibodies are produced by transferring one or more CDRs from the heavy and light variable chains of a mouse (or other species) immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found, for example, in Jones et al., Nature (1986); 321: 522 and Singer et al., J. Immunol., (1993); 150: 2844.

In addition, chimeric antibodies can be obtained by splicing the genes from a mouse antibody molecule with appropriate antigen specificity together with genes from a human antibody molecule of appropriate biological specificity; see, for example, Takeda et al., Nature (1985); 314: 544-546. A chimeric antibody is one in which different portions are derived from different animal species.

The phrase “specifically binds” or “specifically immunoreactive with,” when referring to an antibody, refers to a binding reaction that is determinative of the presence of a protein in a heterogeneous population of proteins and other biologics. Thus, under designated immunoassay conditions, the specified antibodies bind to a particular protein at least two times the background and do not substantially bind in a significant amount to other proteins present in the sample. Typically a specific or selective reaction will be at least twice background signal or noise and more typically more than 10 to 100 times background. Specific binding to an antibody under such conditions may require an antibody that is selected for its specificity for a particular protein.

The present invention also includes a method for detecting the presence of assemblies of amyloid β protein in a sample taken from a subject by contacting a sample with one of the antibodies as discussed herein and detecting binding of the antibody. The sample may be, for example, serum, blood, cerebrospinal fluid (CSF), or brain tissue.

The present invention includes a method of disrupting memory of learned behavior in a non-human mammal by administering an amyloid-β protein assembly intracranially. Intracranial administration includes, for example, intracerebral or intracerebroventricular (ICV) administration. The non-human mammal may be a rat, mouse, dog, or primate. The mammal may serve as an animal model system for a cognitive disorder, including, but not limited to, Alzheimer's Disease. The mammal may demonstrate cognitive deficits consistent with presymptomatic or early Alzheimer's disease. The amyloid-β protein assembly may be made up of six amyloid-β protein dimers, four amyloid-β protein trimers, three amyloid-β protein tetramers, or two amyloid-β protein hexamers.

The present invention includes an animal model for a cognitive disorder, the animal model being a non-human mammal having amyloid-β protein assemblies administered intracranially. The non-human mammal may be a rat, mouse, dog, or primate. The mammal may demonstrate disruption of complex learned behaviors. The mammal may demonstrate cognitive deficits consistent with presymptomatic or early Alzheimer's disease.

Intracranial delivery of assemblies of amyloid β protein may be by any of a wide variety of means. For example, intracranial delivery may be accomplished by oral, subcutaneaous, intraperitoneal, intravenous, and/or intrathecal administration. Delivery may be by local delivery or injection. Delivery may be pump or extended release composition. Intracranial administration may include, for example, intracerebral or intracerebroventricular administration. Assemblies of amyloid y may be delivered to an animal, for example, a vertebrate animal, including a mammal. Mammals include, for example, a rodent, including, but not limited to, a mouse or a rat, a dog, a non-human primate, or other non-human mammals.

Assemblies of amyloid β may be administered in a wide range of concentrations. For example, assemblies of amyloid β may be administered at concentrations that are higher than the concentration of assemblies of amyloid β found in brain or cerebrospinal fluid (CSF); assemblies of amyloid β may be administered at concentrations that are the same or similar to the concentration of assemblies of amyloid β found in brain or CSF; or assemblies of amyloid β may be administered at concentrations that are less than the concentration of assemblies of amyloid β found in brain or CSF.

The present invention includes a method of screening for an agent effective for the treatment of a cognitive disorder by administering an agent to a first animal to which isolated soluble amyloid-β protein assemblies having one or more detergent-stable oligomers of amyloid-β protein have been intracranially administered; measuring cognitive function of said first animal; comparing the cognitive function of said first animal to the cognitive function of a second animal treated in the same fashion except no agent was administered; wherein an improvement in the cognitive function of said first animal compared to the cognitive function of said second animal indicates said agent is effective for the treatment of a cognitive disorder. The present invention also includes agents identified by the screening methods described herein and methods of treatment that include the administration of such agents.

The present invention includes a method of screening for agents that inhibit or prevent the assembly of monomers of amyloid β protein into a detergent-stable oligomer of amyloid-β proteins, agents identified by such screening methods, and methods of treatment that include the administration of such agents.

The present invention also includes agents that inhibit or prevent the assembly of one or more detergent-stable oligomers of amyloid β protein into a soluble amyloid-β protein assembly, agents identified by such a screening method, and methods of treatment that include the administration of such agents.

An agent may be administered by any of a wide variety of means. For example, an agent may be delivered orally, subcutaneaously, intramuscularly, intravenously, intrathecally, and/or intracranially. Delivery may be by local delivery or injection. Delivery may be by pump or extended release composition. An agent may be delivered prior to, during, and/or after delivery of another therapeutic agent. An agent may be delivered prior to during, and/or after the measurement of cognitive functioning. One or more agents may be administered.

As used herein, “treating” a condition or a subject includes therapeutic, prophylactic, and/or diagnostic treatments. Treatment can be initiated before, during, and/or after the development of the condition to be treated.

Suitable agents include any of a wide variety of molecules, including, but not limited to, polypeptides, nucleic acids, antibodies, antisense molecules, ribozymes, small chemical molecules, and the like.

The present invention includes methods of detecting a cognitive disorder in a subject by detecting soluble amyloid-β protein assemblies having one or more detergent-stable oligomers of amyloid-β protein in a fluid or tissue sample taken from the subject.

The present invention includes methods of detecting presymptomatic Alzheimer's disease in a subject by detecting soluble amyloid-β protein assemblies having one or more detergent-stable oligomers of amyloid-β protein in a fluid or tissue sample taken from the subject.

The present invention includes methods for assaying the effects of soluble oligomers of the amyloid β protein on cognitive function by administering a soluble amyloid-β protein assembly having one or more detergent-stable oligomers of amyloid-β protein intracranially into an animal and measuring cognitive function to determine the long-term disruption of cognitive behavior. In some embodiments, the disruption of cognitive behavior of the animal may be compared to the disruption of cognitive behavior of another animal treated in the same fashion except saline rather a soluble amyloid-β protein assembly having one or more one detergent-stable oligomers of amyloid-β protein administered intracranially.

The present invention includes methods for isolating soluble amyloid-O protein assemblies having one or more one detergent-stable oligomers of amyloid-β protein by homogenizing neuronal tissue in a lysis buffer, size fractionating amyloid-β protein assemblies, and isolating an amyloid-β protein assembly of a desired size. For example, about 100 to 200 mg of brain tissue may be harvested in 500 ml of a lysis solution containing 50 mM Tris-HCl (pH 7.6), 0.01% NP-40, 150 mM NaCl, 2 mM EDTA, 0.1% SDS with 1 mM phenylmethylsulfonyl fluoride (PMSF) in the presence of a protease inhibitor cocktail (Sigma). Then, the lysate may be mechanically homogenized using a 1 ml syringe and needle, gage 20, repeating ten times and centrifuged for 90 minutes at 13,000 rpm. The Aβ assemblies of different sizes may be separated by size-exclusion chromatography on Tricorn Superdex® 75 columns (Amersham Life Sciences, Piscataway, N.J., USA) run at a flow rate of 1 ml/minutes in 50 mM Ammonium Acetate, pH 8.5. Then eluted proteins may be concentrated by evaporation using a vacuum system (SpeedVac®, Savant Technologies, USA).

The present invention is illustrated by the following examples. It is to be understood that the particular examples, materials, amounts, and procedures are to be interpreted broadly in accordance with the scope and spirit of the invention as set forth herein. All headings are for the convenience of the reader and should not be used to limit the meaning of the text that follows the heading, unless so specified.

### EXAMPLES

### Example 1

### Specific Amyloid-β Protein Assembly in the Brain Impairs Cognitive Function

Cognitive function often declines with age and is believed to deteriorate initially because of changes in synaptic function rather than loss of neurons. Some individuals progress to develop Alzheimer's disease (AD) with neurodegeneration. In this example Tg2576 mice were used to investigate the cause of cognitive decline in the absence of neurodegeneration or amyloid-β (Aβ) protein amyloidosis. Strong correlations were found between memory deficits and extracellular accumulation of a 56-kD soluble Aβ assembly, called Aβ*56 (Aβ star 56), which disrupts cognitive function when injected directly into healthy rats. This example demonstrates that Aβ*56 disrupts cognitive function independently of plaques or neuronal loss, and contributes to cognitive deficits associated with AD.

Age-related cognitive decline (ARCD) occurs in many mammalian species (F. I. Craik, in Handbook of the Psychology of Aging J. E. Birren, K. Schall, Eds. (Van Nostrand-Reinhold, New York, 1977) pp. 384-420; and Gallagher and Rapp, Annu Rev Psychol 48, 339 (1997), including humans, and appears to result from synaptic dysfunction (Morrison and H of, Science 278, 412 (1997). Poor cognitive function can predict AD up to fifteen years before diagnosis (Kawas et al., Neurology 60, 1089 (2003), and non-demented individuals at risk genetically for AD show abnormalities in functional brain imaging tests (Small et al., Proc Natl Acad Sci USA 97, 6037 (2000); and Bookheimer et al., N Engl J Med 343, 450 (2000)). These and other studies imply that AD has an insidious onset, which blurs the boundary between ARCD and AD (Albert and Drachman, Neurology 55, 166 (2000)).

Tg2576 mice express a human amyloid-β precursor protein (APP) variant linked to AD and develop many neuropathological features of AD, including amyloid plaques containing Aβ, dystrophic neurites and inflammatory changes (Hsiao et al., Science 274, 99 (1996); and Benzing et al., Neurobiol Aging 20, 581 (1999)), but lack neurofibrillary tangles, significant neuronal loss or gross atrophy (Irizarry et al., J Neuropathol Exp Neurol 56, 965 (1997)). Young Tg2576 mice (less than six months) have normal memory and lack neuropathology, middle-aged mice (aged six to fourteen months) develop memory deficits without neuronal loss, and old mice (greater than fourteen months of age) form abundant neuritic plaques associated with minimal neuronal loss (Hsiao et al., Science 274, 99 (1996); Irizarry et al., J Neuropathol Exp Neurol 56, 965 (1997); Kawarabayashi et al., Journal of Neuroscience 21, 372 (2001); Westerman et al., J Neurosci 22, 1858 (2002); and Urbanc et al., Proc Natl Acad Sci USA 99, 13990 (2002)). Tg2576 mice are therefore a good model to study pre-clinical stages of AD, prior to the diagnosis of dementia or the onset of neuronal loss.

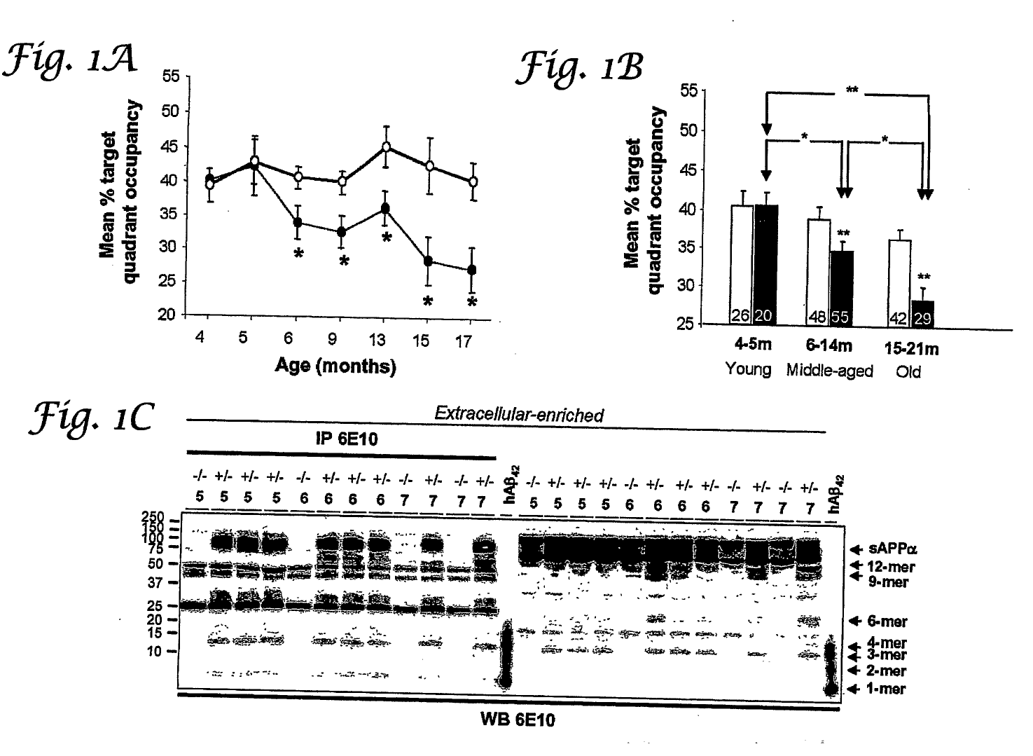

In Tg2576 mice, as in other APP transgenic mice, there is strong evidence that Aβ is responsible for age-related deterioration in cognitive function (Westerman et al., J Neurosci 22, 1858 (2002); Janus et al., Nature 408, 979 (2000); and Chen et al., Nature 408, 975 (2000)). However, there are several paradoxical findings about the relationship between Aβ and cognitive decline that suggest a complex role of Aβ in cognitive impairment. For example, spatial reference memory in Tg2576 mice declines modestly but significantly at six months and remains stable for 7 to 8 months (FIGS. 1A and 1B). However, no candidate Aβ species measured to date corresponds with the decline in memory observed at six months and the cognitive stability observed thereafter (see Table 2). Thus, the enigma that a rapidly increasing amount of Aβ, the molecule believed to be responsible for cognitive impairment, is associated with no change in cognitive function. One solution to this conundrum is to posit the existence of soluble Aβ assemblies that disrupt cognition (Hsia et al., Proc Natl Acad Sci USA 96, 3228 (1999); Ashe, Learn Mem 8, 301 (2001); and Klein et al., Trends Neurosci 24, 219 (2001)). With this example, Aβ*(Aβ star) was identified in Tg2576 mice.

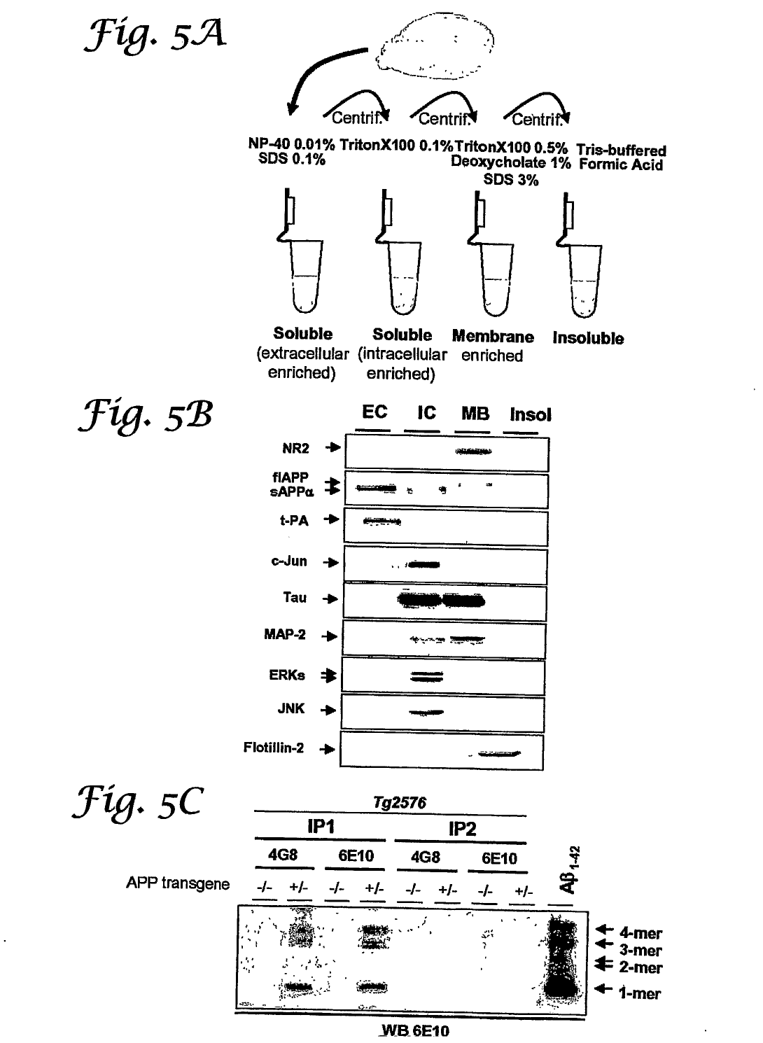

A major challenge in analyzing Aβ in the brain lies in reliably separating Aβ from various cellular compartments (for example, extracellular, intracellular, and membrane associated). This obstacle was overcome by developing an extraction procedure that separates proteins in known cellular compartments with high fidelity (FIG. 5A). The extraction method used in this example allowed the quantification and comparison of up to four different pools of transgene-derived Aβ species.

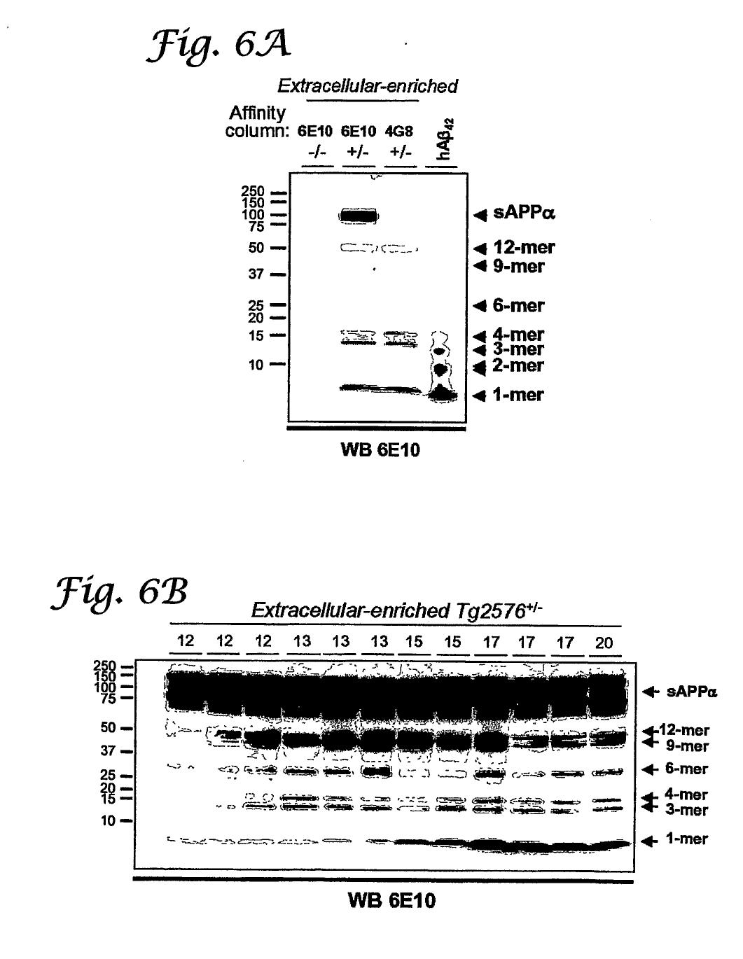

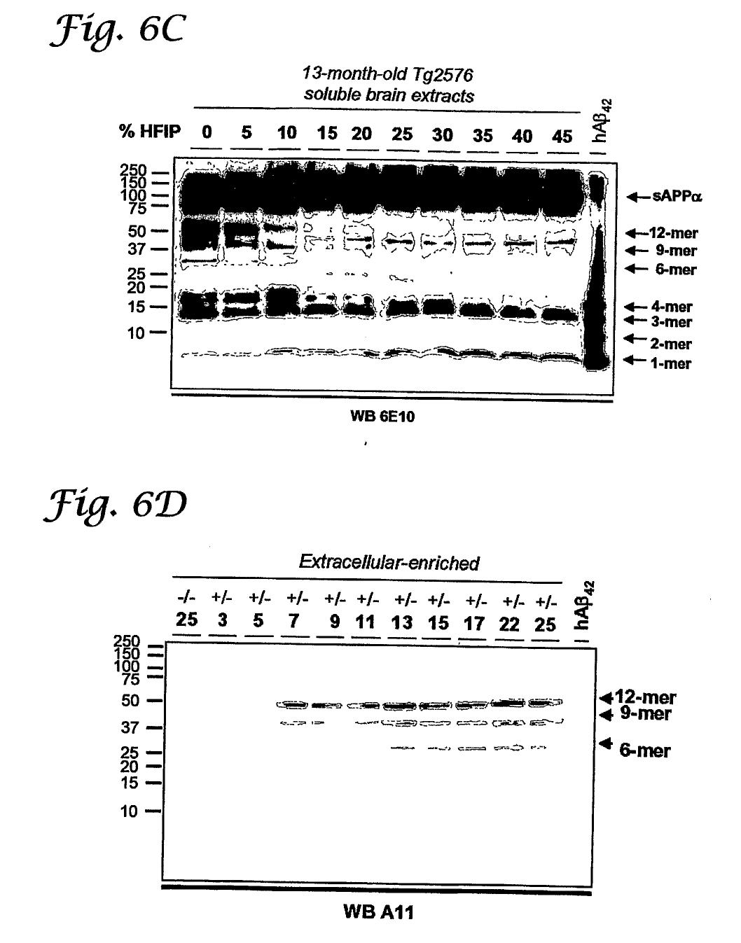

To resolve the paradox of a mismatch between Aβ levels and memory deficits, the extraction procedure was used to search for Aβ* in Tg2576 mice between four and twenty-five months. Aβ* molecules were required to satisfy the following two criteria: one) their appearance coincides with memory loss at six months; and two) their levels remain stable in middle-aged mice (aged six to fourteen months). By immunoblotting immunoglobulin-depleted brain extracts, a set of Aβ protein assemblies was found in the soluble, extracellular-enriched fraction from six-month mice (FIG. 1C). Besides a faint band corresponding to A13 monomers, there were bands co-migrating with trimeric (14-kD), hexameric (27-kD), nonameric (40-kD) and dodecameric (56-kD) Aβ assemblies. Interestingly, these species represent multiples of trimeric Aβ oligomers, with high molecular weight (HMW) assemblies (greater than 20 kDa) appearing in mice of six or more months of age. Identical bands were detected using 6E10 and 4G8 antibodies, excluding the possibility that they represented degradation products of soluble APP (FIG. 6A).

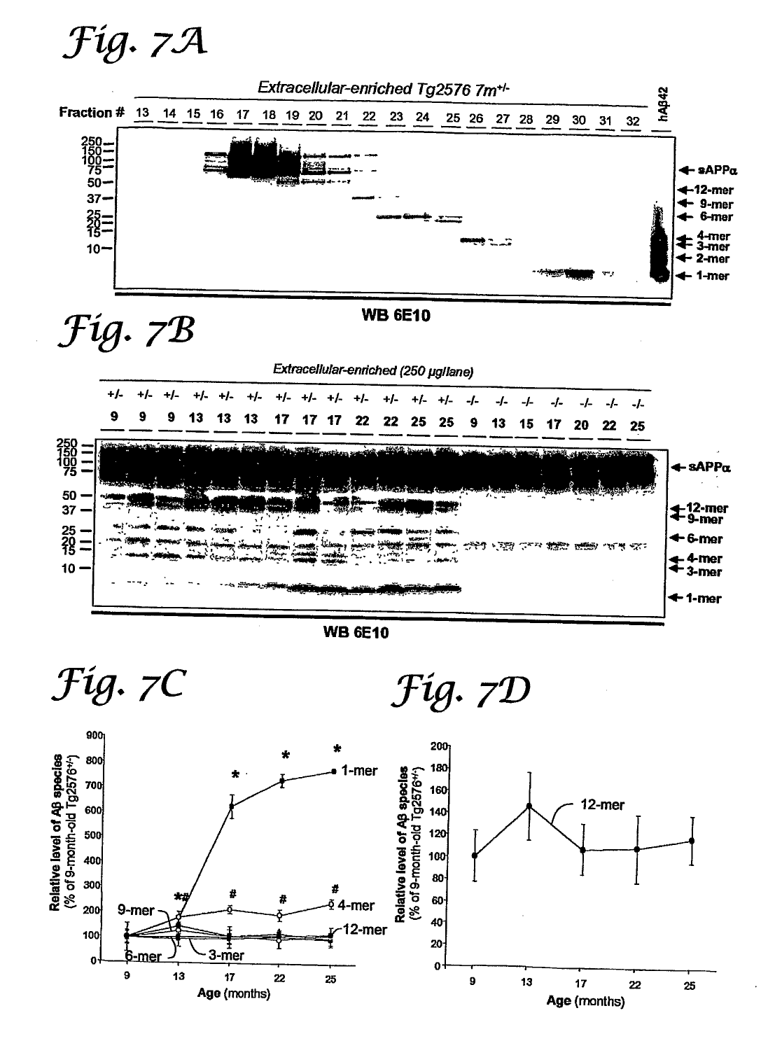

Although this result suggests that aging induces trimers to associate and form HMW assemblies, the possibility that the HMW assemblies represent Aβ tightly bound to other proteins cannot be excluded. However, this is unlikely based upon their stability and immunospecificity. First, the possibility that they would be disrupted by urea, a common denaturant of globular proteins (Gordon et al. J Biol Chem 243, 5663 (1968)), was considered. All Aβ oligomers were detected in 8M urea-containing SDS-PAGE (FIG. 6B), arguing against the presence of a globular protein. In contrast, when exposed to ≧15% hexafluoroisopropanol (HFIP), all but trimers and, to a lesser extent, tetramers (which form at 13 months) disassembled (FIG. 6C), suggesting that the HMW complexes are held together by hydrogen, not covalent, bonds. The resistance of trimers to dissociation in greater than 25% HFIP supports the conclusion that trimers are the fundamental assembly unit. Second, the HMW complexes are recognized by the A11 antiserum (FIG. 6D), which specifically detects soluble Aβ assemblies that are distinct from Aβ fibrils in AD brain (Kayed et al., Science 300, 486 (2003)). This antiserum revealed HMW assemblies, but not trimers, in soluble, extracellular-enriched fractions, implying that the HMW complexes are soluble Aβ oligomers and confirming previous observations that A11 reacts only with Aβ oligomers larger than tetramers (Kayed et al., Science 300, 486 (2003)). Finally, since it is possible that trimers might be fragments of HMW complexes released during the sample preparation or gel fractionation procedures, or HMW assemblies might be artificially generated during the biochemical procedures, the native size of soluble Aβ assemblies was examined under non-denaturing conditions. Soluble, extracellular-enriched extracts were fractionated by size-exclusion chromatography (SEC) and subjected to SDS-PAGE (FIG. 7A). High and low molecular weight (MW) Aβ assemblies were found in the collected fractions, at the appropriate intervals. Taken together, these results indicate that Aβ in six-month Tg2576 mice forms a ladder of stable, soluble, physiological assemblies comprised of trimers and multiples of trimers.

Trimers and hexamers were excluded as components of Aβ*, because they were present prior to memory impairment (less than six months). However, a 56-kD band, corresponding to dodecamers, appeared at six months, along with lesser quantities of nonamers (FIG. 1C). The mean levels of the trimeric, hexameric, nonameric and dodecameric Aβ assemblies in Tg2576 mice up to twenty-five months remained stable throughout life, although there was considerable variability between animals of the same age (FIGS. 7B-D). Because dodecamers and nonamers appeared at six months and remained stable throughout life, they fulfilled the criteria for being designated as Aβ*. No Aβ assemblies were found in old mice corresponding to the second drop in memory function at fifteen months (FIGS. 1A and 1B); by this age it is possible that the abundant plaques with prominent dystrophic neurites disrupt synaptic function sufficiently to impair memory further (Stern et al., J Neurosci 24, 4535 (2004)).

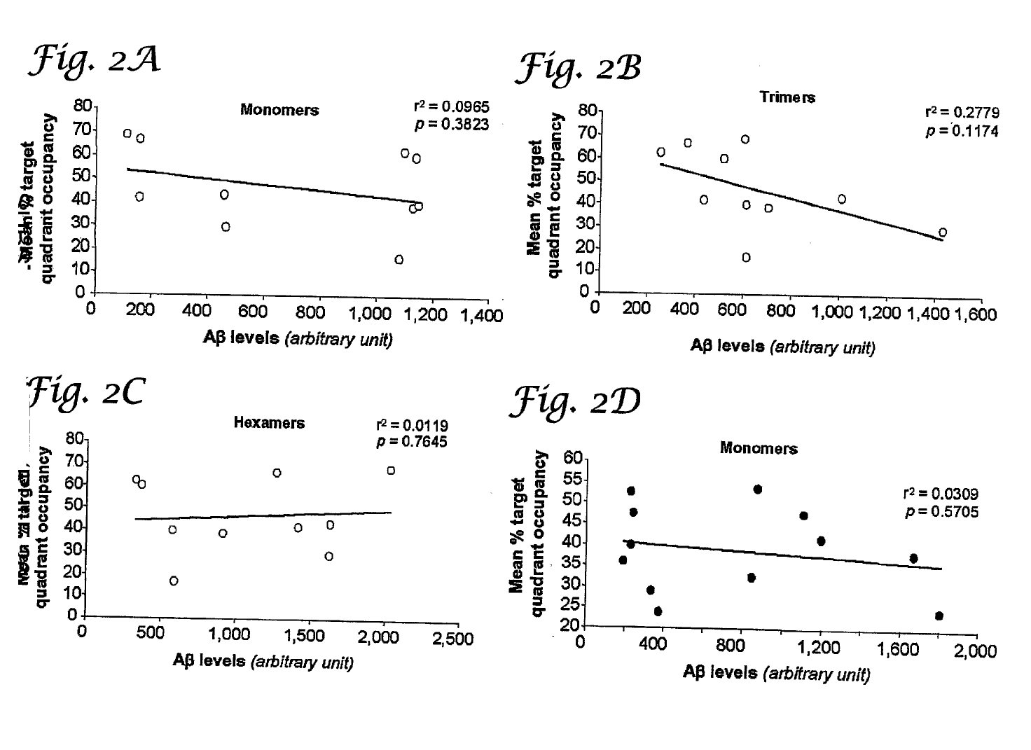

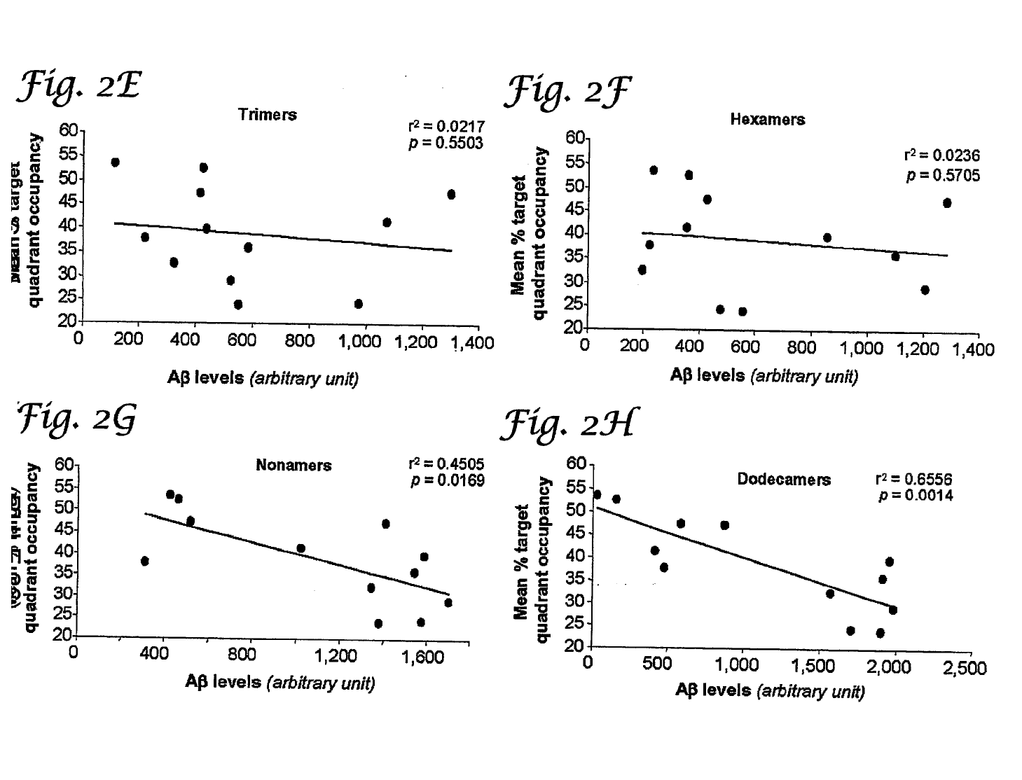

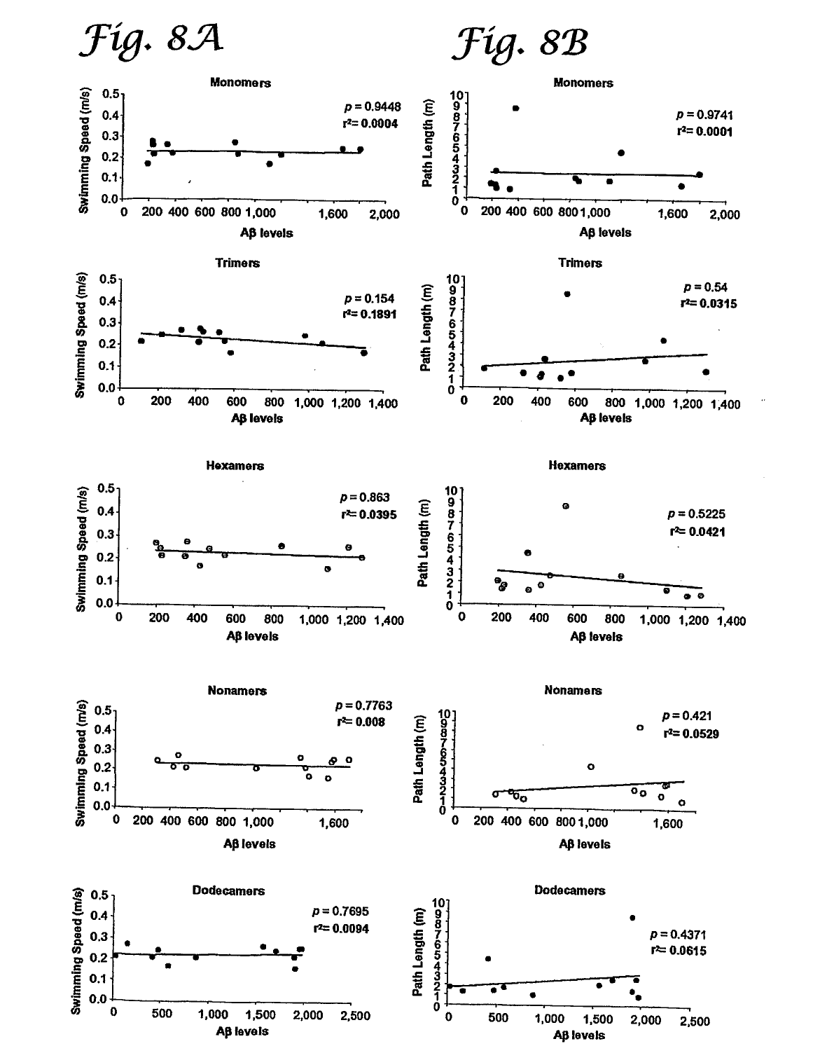

The variability in levels of Aβ assemblies between animals of the same age provided an opportunity to examine correlations between the different Aβ oligomers and memory impairment, which were assessed by comparing spatial memory and soluble Aβ species in two groups of five- and six-month-old Tg2576 mice (FIG. 2). Monomers, trimers and hexamers of Aβ did not correlate significantly with performance in five- or six-month mice (FIGS. 2A-F). However, the nonamers and dodecamers, which were present in six-month but not five-month animals, correlated significantly with performance in an inverse relationship (FIGS. 2G and 2H). Importantly, the appearance of the 56-kD Aβ species (putative dodecamers) at six months was not associated with deficits in the cued phase of water maze testing, indicating that this Aβ species is not sufficient to impair sensory motor function or to alter motivation, attention or activity levels. There were no correlations between the levels of any Aβ oligomers and performance in the cued phase of the water maze test, further arguing against their effects on non-cognitive aspects of behavior (FIG. 8).

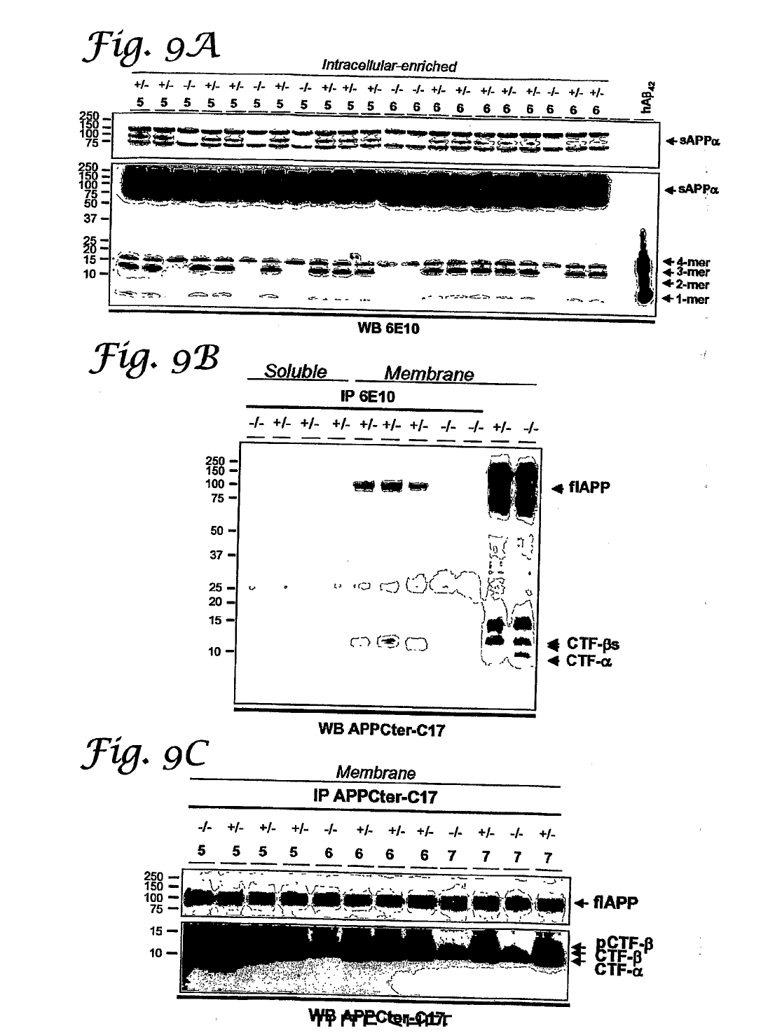

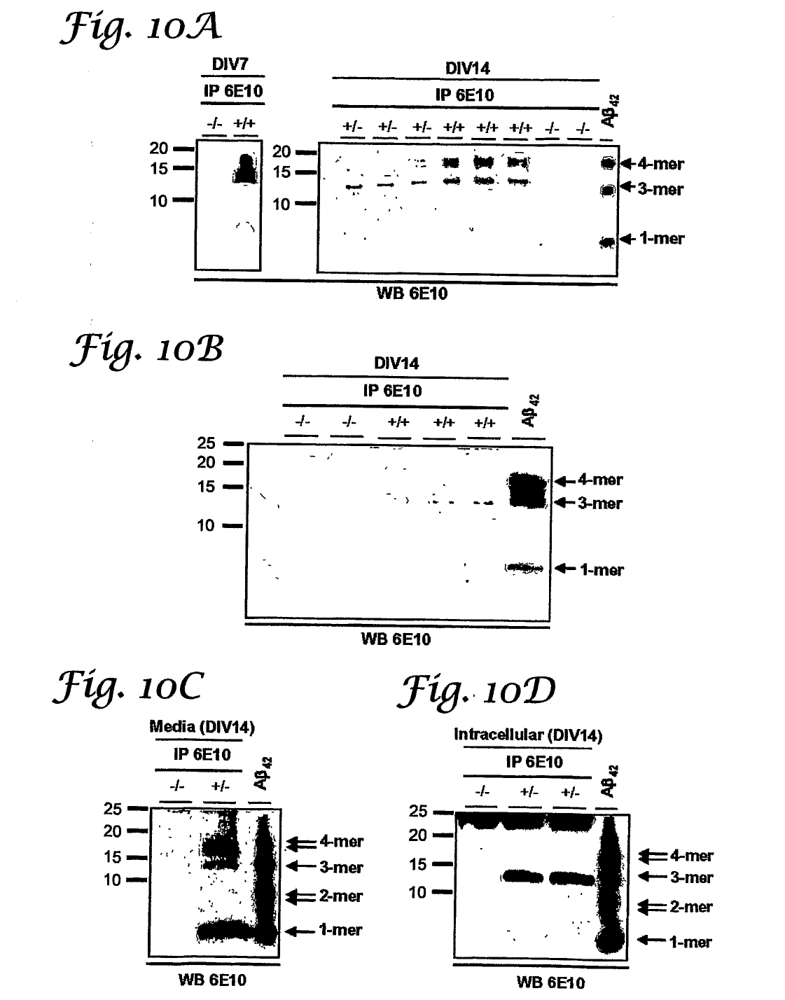

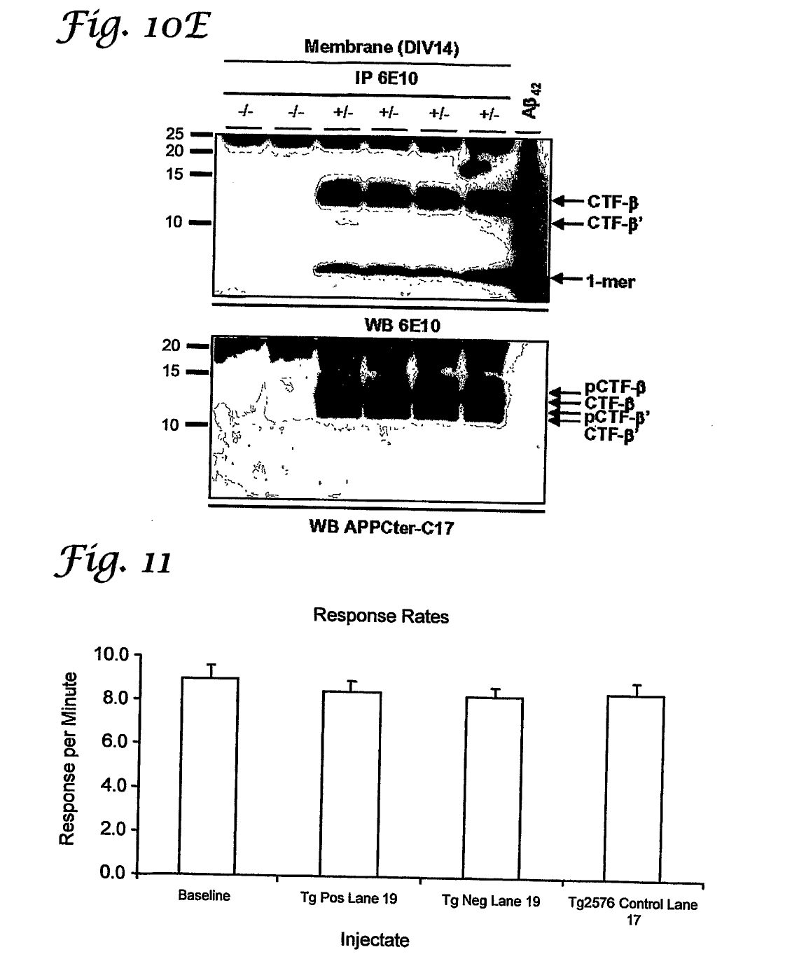

Because intracellular Aβ has been proposed to disrupt memory in 3×Tg-AD (Billings et al., Neuron 45, 675 (2005)), additional analyses was performed to evaluate the potential accumulation of Aβ species within brain cells. Only trimeric and monomeric Aβ species were detected in the soluble, intracellular-enriched fraction, with no modulation between five and six months (FIG. 9A). Full-length APP and C-terminal fragments (CTFs) in membrane-associated fractions were also examined, and no change in the levels of APP, CTF-βs or CTF-α was found (FIGS. 9B and 9C). In cytosolic extracts of cultured primary Tg2576 cortical neurons, monomers and trimers were the only Aβ species detected (FIG. 10), supporting the in vivo findings. Thus, no intracellular or membrane-associated Aβ species or CTFs correlating with the onset of memory deficits were found in six-month Tg2576 mice.

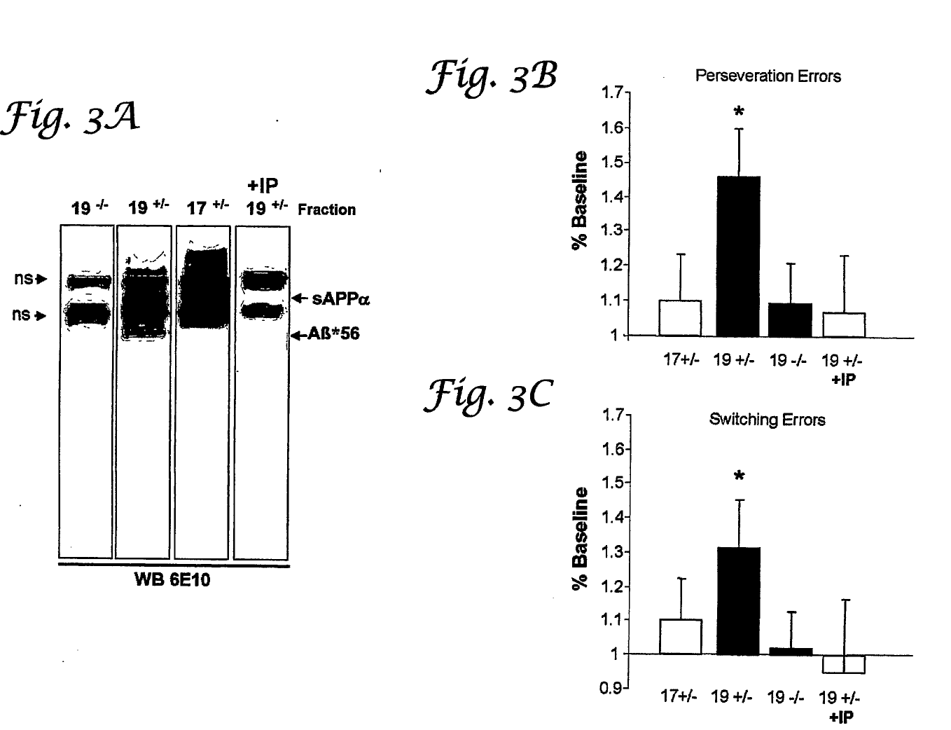

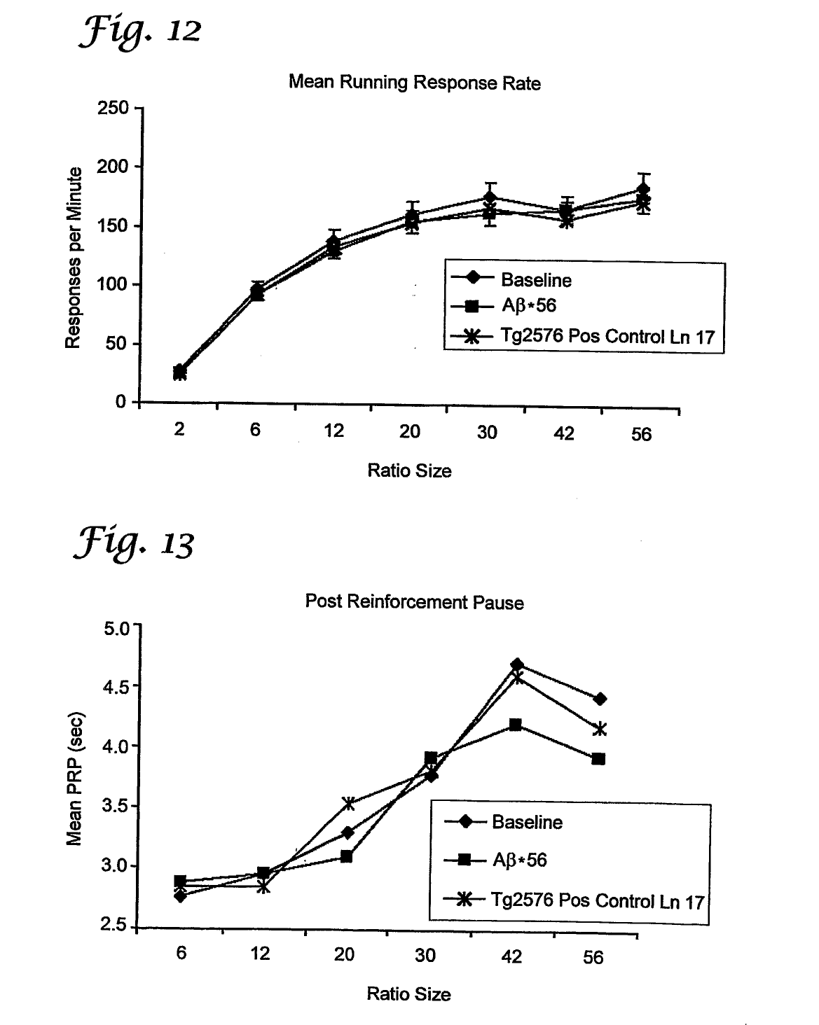

The observations that the 56-kD Aβ assembly appears at six months when memory declines, that its levels are stable in aging mice, that it is more abundant than the 40-kD Aβ complex, and that it correlates most strongly with memory impairment suggested that it was a likely candidate for Aβ*. To determine whether the 56-kD Aβ assembly directly disrupts cognitive function, the 56-kD Aβ assembly was isolated from all other Aβ species and its effect assayed in rats using the Alternating Lever Cyclic Ratio (ALCR) procedure, a test of executive cognitive function and reference memory function, which was previously proven to be sensitive to the behavioral effects of Aβ oligomers secreted by Chinese hamster ovary (CHO) cells (Cleary et al., Nat Neurosci 8, 79 (2005)). In the ALCR paradigm, substances which disrupt cognitive function produce increases in switching and perseveration errors that can be dissociated from effects on motivation and activity levels. To isolate the 56-kD Aβ assembly, proteins were fractionated from soluble, extracellular-enriched brain extracts of seven-month Tg2576 mice and non-transgenic littermates by SEC (FIG. 7), and injected selected fractions into the lateral cerebral ventricles of ALCR-trained rats. Fraction 19+/− containing the 56-kD Aβ assembly was found to significantly increase switching and perseveration errors measured two hours after the injections; increases were not found with the corresponding fraction 19−/− from a non-transgenic littermate or fraction 19+/− after immunoprecipitation with 6E10 (FIGS. 3B and 3C). The increase in errors cannot be attributed to changes in motivation or activity levels because the response rates were unaffected (FIG. 11 and FIG. 12), in keeping with the preservation of performance during the cued phase of water maze testing in Tg2576 mice. The data show that the effects of the 56-kD Aβ assembly were on cognitive function rather than on non-cognitive aspects of behavior.

To exclude the possibility that cognitive function was disrupted by 6E10-reactive proteins greater than 60-kD in fraction 19+/−, fraction 17+/− containing HMW 6E10-reactive proteins, but lacking the 56-kD protein, was tested and no significant increase in errors was found. In addition, fraction 26+/− containing trimers did not significantly increase errors, consistent with the failure of trimers to correlate significantly with memory impairment. Interestingly, the increases in errors as a result of the 56-kD Aβ assembly were transient; that is, they were not significantly increased when assessed the day after the ICV injection, following a time course similar to that of Aβ oligomers generated by CHO cells (Cleary et al., Nat Neurosci 8, 79 (2005)). These results indicate that an extracellular 56-kD assembly of soluble A13, herein named Aβ*56, directly and specifically disrupts cognitive function in healthy rats.

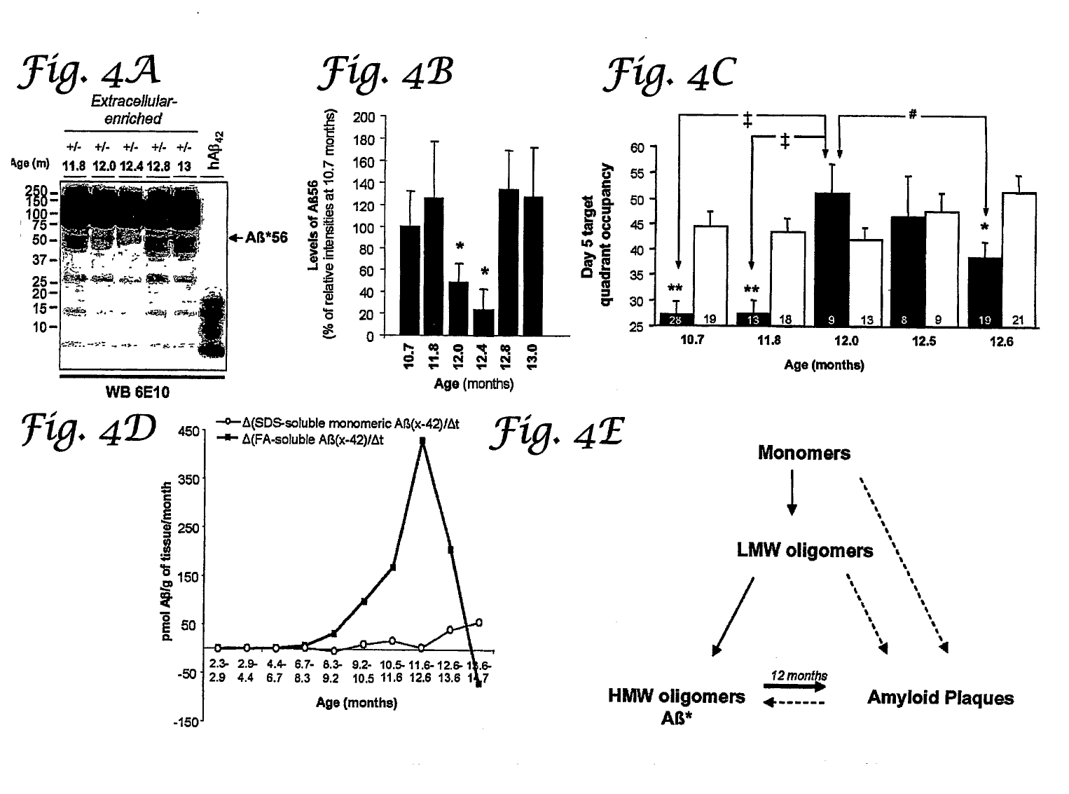

To further link Aβ*56 to memory loss, it was asked whether eliminating it would restore memory. Lacking specific antibodies targeting Aβ*56, an experiment of nature was utilized to examine the consequences on memory of removing it from the brain. A transient disappearance of Aβ*56 was found between 12.0 and 12.4 months of age in Tg2576 mice (FIGS. 4A and 4B). To investigate the effects on memory of eliminating Aβ*56, spatial memory was tested in five separate groups of Tg2576 mice whose ages spanned an interval surrounding twelve months. Although the performance of Tg2576 mice at 10.7 and 11.8 months was lower than in non-transgenic (non-Tg) littermates, it improved significantly in 12.0- and 12.5-month animals and was similar to that of non-Tg littermates (FIG. 4C). However, this restoration of memory was brief. With the reappearance of Aβ*56, the performance of 12.6-month Tg2576 mice declined and was significantly lower than in non-Tg littermates. Thus, the elimination of Aβ*56 restored spatial reference memory, indicating that Aβ*56 specifically causes memory loss in Tg2576 mice.

Interestingly, when the rates of change of SDS-soluble and SDS insoluble Aβ(x-42) were calculated using previously published results (Kawarabayashi et al., Journal of Neuroscience 21, 372 (2001)), it was found that a brief dip in Aβ*56 coincided with the peak rate of accumulation of SDS-insoluble Aβ(x-42) occurring between 11.6 and 12.6 months of age (FIG. 4D), when mature amyloid plaques consistently appear (Kawarabayashi et al., Journal of Neuroscience 21, 372 (2001)). This observation may be explained by soluble Aβ assemblies and fibrillar Aβ existing in a dynamic equilibrium with each other or competing for the same pool of monomeric Aβ (FIG. 4E), indicating that very high rates of deposition of fibrillar Aβ create a “sink” leading to reduced levels of Aβ*56.

Aβ*56 may elucidate the functional significance of a soluble putative Aβ dodecamer in AD brain which is recognized by antibodies to Aβ-derived diffusible ligands (ADDLs) (Gong et al., Proc Natl Acad Sci USA 100, 10417 (2003)). However, in Tg2576 mice, antibodies raised against ADDLs detect a 20- to 100-fold increase in the cortical signal between 13 and 17 months (Chang et al., J Mol Neurosci 20, 305 (2003)), and a 130-fold increase in the hippocampal signal between 9 and 20 months. Since the levels of Aβ*56 do not change appreciably with age, these results indicate that anti-ADDL antibodies do not specifically detect Aβ*56. Aβ*56 impairs memory by inducing transient physiological, rather than permanent neuropathological, alterations of the brain, as inferred by the transient effects following injections into rats and the interlude of normal memory in 12-month Tg2576 mice that occurs when Aβ*56 levels dip and mature plaques appear. Intriguingly, the coincidence of improved memory and the appearance of plaques suggests that amyloid deposition, at least in the earliest stages, may protect the brain from the detrimental effects of Aβ*. This could explain why some cognitively intact individuals have high plaque loads (Crystal et al., Neurology 38, 1682 (1988)).

The accumulation of insoluble aggregates consisting of proteins such as tau, huntingtin, prion protein, ataxin, and Aβ occurs in many neurodegenerative disorders. These aggregates often define the disorders neuropathologically, but their relative contribution to disease symptoms compared to other, hypothetical, intermediate protein assemblies is controversial (Orr, Nature 431, 747 (2004); Santacruz et al., Science 309, 476 (2005); and Duff and Planel, Nature Medicine 11, 826 (2005)), and the identity of the theoretical intermediates has been elusive. The present discovery, that Aβ*56 is responsible for memory loss in plaqueforming Tg2576 mice, and causes cognitive deficits when injected directly into healthy rats, sets a precedent for identifying other “star” proteins inducing brain dysfunction; such as, for example, a tau* (Santacruz et al., Science 309, 476 (2005)). That Aβ* is a highly specific form of Aβ offers the potential for developing precise diagnostic methods to detect its correlate in humans with pre-clinical AD, opening the possibility of targeting Aβ* and aborting the disease before permanent structural changes have developed.

Methods

Transgenic animals. Tg2576 mice (Hsiao et al., Science 274, 99 (1996)) were the offspring of mice backcrossed successively to B6SJLF1 breeders, except for mice used in the behavioral and biochemical experiments shown in FIGS. 4A-4C, which were in the 129FVBF1 strain background.

Antibodies. The following primary antibodies were used: 6E10 and 4G8 [1:100-10,000 dilution] (Signet Laboratories, USA), R1282 [1:75 dilution] (Walsh et al., Nature 416, 535 (2002)), R1736 [1:1000 dilution] (Haass et al., Nature 357, 500 (1992)), FCA3542(Barelli et al., Mol Med 3, 695 (1997)), APPCter-C17 [1:5000 dilution] (Sergeant et al., J Neurochem 81, 663 (2002)), anti-Flotillin-2, anti-ERKs, anti-JNK and anti-c-Jun [all 1:200 dilution] (Santa Cruz Biotechnology, Inc., USA), Tau-5 [1:1,000 dilution] (Biosource International, USA), anti-MAP-2 [1:200 dilution] and anti-Actin [1:250] (Sigma) and anti-t-PA (American Diagnostica Inc., USA). The A11 anti-oligomer antibody [1:5,000 dilution] (Kayed et al., Science 300, 486 (2003)) was detected with a biotinylated anti-rabbit antibody [1:2,000,000 dilution] (Vector Laboratories) and ExtrAvidin® [1:5,000 dilution] (Sigma).

Morris water maze behavioral test. Spatial reference memory was assessed using a modified version of the Morris water maze (Westerman et al., J Neurosci 22, 1858 (2002)). Testing was tailored for Tg2576 transgene positive and negative mice in each background strain, since 129FVBF1 mice learn more rapidly than B6SJL mice. B6SJLTg2576 transgene positive and negative mice received visible platform training for three days, eight trials per day, followed by hidden platform training for nine days, four trials per day. Three probe trials were performed twenty hours after twelve, twenty-four, and thirty-six training trials, and the mean % target quadrant occupancy for the three probe trials, was calculated. 129FVBF1-Tg2576 transgene positive and negative mice received visible platform training for three days, six trials per day, followed by hidden platform training for six days, four trials per day. Probe trials performed twenty hours after four, eight, sixteen, and twenty-four training trials lasted sixty seconds, but % target quadrant occupancy was calculated using the first thirty seconds because the 129FVBF1 mice exhibited extinction. The probe trial following sixteen training trials (Day 5 target quadrant occupancy) was determined to be the most sensitive to the effect of transgene on performance across the age range tested. To coordinate the timing of the assessment of memory with the brief dip in Aβ*56 levels, we used this probe trial to measure retention of spatial memory in 129FVBF1-Tg2576 mice.

Alternating Lever Cyclic Ratio (ALCR) Procedure.

Subjects. Thirty-eight male Sprague-Dawley rats, approximately 120 days old and weighing 250-300 grams (g) at the beginning of the experiment, were housed individually with free access to water in a temperature and humidity controlled environment. Rats were maintained at 90% of their free-feeding weights during the experimental studies. During the course of the study, four rats' data were removed due to illness from blocked cannulae.

Apparatus. Behavioral training and testing was conducted in two-lever rat test chambers (model EIO, Coulbourn Instruments, Inc.) enclosed in sound attenuating compartments. Each food reinforcer consisted of a 45 milligram (mg) pellet (F0021, Bioserv Holton Ind., Frenchtown, N.J.) delivered into a tray situated midway between the levers. A food tray light flash and an audible pellet dispenser click signaled food delivery. Control of experimental contingencies and data collection was accomplished using computer MED PC computer software and interface (Med Associates, Fairfield N.J.).

Alternating Lever Cyclic Ratio (ALCR) behavioral procedures and testing. In the ALCR task, subjects learn a sequence of lever-pressing requirements for food reinforcement in a two-lever experimental chamber. Rats must alternate responding between the two levers, switching to the other lever after pressing enough to get a food pellet. The number of presses required for each food reinforcer varies from low (for example, two) to high (for example, fifty-six), incorporating intermediate values based on the quadratic function [x²−x]. One response cycle is an entire ascending and descending sequence of these response requirements (2, 6, 12, 20, 30, 42, 56, 56, 42, 30, 20, 12, 6, and 2 presses per food reinforcer). Six cycles are presented during each session. Thus, the subject alternates responding on the two levers, with an increasing, and then a decreasing, response per reinforcement ratio, six times per session. Errors occur when the subject perseveres on a lever after reinforcement, i.e., does not alternate (perseveration error), or when a subject switches levers before completing the response requirement on that lever (switching error). Perseveration errors are accumulated until the subject presses the correct lever, while switching errors are counted as a single occurrence for each premature switch to the incorrect lever.

In addition to errors, dependent variables relevant to activity levels (Overall Response Rate and Running Response Rate) and the subjects' abilities to track the size of the current work requirement (Post Reinforcement Pause and relationship of Response Rate to Lever Press Requirement) are also collected. Overall Response Rate is calculated as lever presses per second over the entire session, while Running Response Rate is calculated as the response rate only during the time the rat is actively engaged in lever-pressing. Post Reinforcement Pause (PRP) is the pause time in seconds that typical occurs following reinforcement. PRP is directly related to the work required (presses) for each reinforcer. Similarly, lever press rates (responses per unit time) are known to vary directly with the presses per reinforcer requirement.

ALCR Training. Behavioral sessions were conducted five to seven days per week. Rats were first trained to press both levers for food reinforcement and subsequently reinforced for a lever press only if they switched levers after each reinforcer delivery. The ratio of required responses per reinforcer delivery was slowly increased to 10:1 across 26 daily sessions. At this point the ALCR was introduced using sequential response per reinforcer ratios of: 1 3 5 8 10 15 15 10 8 5 3 1. This cycle repeated six times during each daily session. The response per reinforcer ratio cycle was slowly increased to the terminal values of: 2 6 12 20 30 42 56 56 42 30 20 12 6 2, required responses per reinforcer, and the cycle repeated six times each session. Sessions ended when the rat completed six cycles or after two hours (h) (mean session time, approximately 40 minutes). Rats received forty sessions of training in the ALCR task prior to surgery.

Baseline errors in the ALCR task. After recovery from surgery, mean baseline error rates (switching and perseveration) in the ALCR task were established for individual rats. To establish baseline error rates, errors were averaged across sessions, with the restriction that the highest and lowest error rate was removed before calculating the mean. During baseline sessions, rats were injected ICV with saline and/or were subjected to ‘sham’ injections under which the entire injection procedure was performed but no injectate was given. To ensure accurate baseline comparisons over the entire course of the study, mean error baselines were re-established after every two or three injections.

ICV injections. During the injection procedure, rats were removed from their home cages, cannula cap stylets were removed, and a 33-gauge internal injection cannula was inserted into the guide cannula. The injection cannula tip extended into the lateral ventricle 0.5 millimeter (mm) past the end of the guide cannula tip. The injection cannula was connected with PE 20 plastic tubing to a 50 microliter (μl) Hamilton syringe containing the injectate. Following the injection, the cannula was capped with a stylet and the rat was placed in a holding box for two hours prior to the ALCR. On non-injection days, rats were subjected to sham injections, under which the same procedure was followed but no injectate was actually given. In addition, 20 μl of saline (0.9%) was injected at least once each week in order to help keep the cannula patent.

Statistics. Perseveration errors and switching errors were not normally distributed. Therefore, a non-parametric matched-pair t-test (Wilcoxon) was used to evaluate within-subject changes in performance from baseline to post-injection within each group. Due to the large differences between switching errors and perseveration errors, these error rates are analyzed separately.

Surgery. Rats were anesthetized using a combination of ketamine (60 milligram/kilogram (mg/kg)) and xylazine (20 mg/kg) and placed in a rat stereotaxic instrument. A 26-gauge guide cannula (Plastic One, Roanoke, Va.) was implanted in either the right or left lateral ventrical. For ICV cannula placement, the stereotaxic coordinates, with the incisor bar set 3.5 millimeter (mm) above the interaural line, were ±1.5 mm lateral and 1.0 posterior to bregma, and 3.5 mm below the surface of the skull. Half the rats received cannulae directed at the right lateral ventricle and half received cannulae directed at the left lateral ventricle. The rats were allowed to recover for five days following surgery.

Cognitive function in rats following injections of SEC fractions into the lateral ventricles was assessed using the ALCR assay (Richardson et al., Brain Res 954, 1 (2002)). Briefly, in the ALCR task rats learn a complex sequence of lever-pressing requirements for food reward. Rats must alternate between two levers, switching levers after pressing one lever enough to get a food pellet. The number of presses required for each food reward proceeds from low (e.g., 2) to high (e.g., 56). One cycle is an entire ascending and descending sequence of these response requirements (e.g., 2, 6, 12, 20, 30, 42, 56, 56, 42, 30, 20, 12, 6, and 2 presses reward). Six full cycles are presented during each session. Based upon response rates and post reinforcement pauses, subjects learn to track the ratio size. Errors are scored when the subject perseveres on a lever after reward, i.e., does not alternate (a perseveration error), or when a subject switches levers before completing the response requirement on that lever (a switching error).

Protein extractions. Hemi-forebrains were harvested in 500 μl of solution containing 50 millimolar (mM) Tris-HCl (pH 7.6), 0.01% NP-40, 150 mM NaCl, 2 mM EDTA, 0.1% SDS, 1 mM phenylmethylsulfonyl fluoride (PMSF), and protease inhibitor cocktail (Sigma). Soluble, extracellular-enriched proteins were collected from mechanically homogenized lysates (1 ml syringe, gauge 20 needle [10 repeats]) following centrifugation for 5 minutes at 3,000 rpm. Cytoplasmic proteins were extracted from cell pellets mechanically dissociated with a micropipettor in 500 μl TNT-buffer (50 mM Tris-HCl [pH 7.6], 150 mM NaCl, 0.1% Triton X-100) following centrifugation for 90 minutes at 13,000 rpm. Membrane-associated proteins were extracted from pellets following gentle agitation on a rotating platform in 500 μl of buffer containing 50 mM Tris-HCl [pH 7.4], 150 mM NaCl, 0.5% Triton X-100, 1 mM EGTA, 3% SDS, 1% deoxycholate, 1 mM PMSF, and protease inhibitor cocktail (Sigma) following centrifugation for 90 minutes at 13,000 rotations per minute (rpm). Insoluble material was incubated with 20 μl 80% formic acid, mechanically dissociated with a micropipette, gently agitated for one hour and buffered with 380 μl IM Tris-HCl, pH 8.0. Samples were centrifuged for 90 minutes at 13,000 rpm and the supernatant was collected for analysis.

Fractions were immunodepleted by sequential incubation for one hour at 4° C. with 40 μl of Protein A-Sepharose, Fast Flow® followed by 40 μl of Protein G-Sepharose, Fast Flow® (Amersham Pharmacia Biotech U.K., Ltd., Little Chalfont, Buckinghamshire, U.K.). All supernatants were clarified by centrifuging for 90 minutes at 13,000 rpm prior to western blot analysis. Protein amounts were determined (BCA Protein Assay, Pierce).

Size-exclusion chromatography (SEC). Protein extracts were loaded on Tricorn Superdex® 75 columns (Amersham Life Sciences, Piscataway, N.J., USA) and run at a flow rate of approximately 0.3 milliliter/minute (ml/min). Fractions of 500 μl of eluate in 50 mM Ammonium Acetate, pH 8.5, were evaporated using a vacuum system (VacuFuge™, Brinkmann-Eppendorf, USA).

Western blots and immunoprecipitations. SDS-PAGE: Pre-cast 10-20% SDS-polyacrylamide Tris-Tricine gels (Bio-Rad) or 16% Tris-Tricine gels in the presence or absence of SDS or Urea 8M were used. 100-250 microgram (μg) of protein per sample was resuspended with 4× Tricine loading buffer. Proteins were transferred to PVDF membranes (Immobilon Psq membrane, Millipore) or 0.2 μm nitrocellulose membranes (Bio-Rad). Membranes were boiled for 10 minutes in PBS and blocked in TBST (Tris-Buffered Saline-Tween®20) containing 5% bovine serum albumin (BSA) plus 5% Top-Block (Sigma), and probed with appropriate antisera/antibodies diluted in 5% BSA-5% Top-Block TBST. Blots were developed with an ECL detection system (Supersignal Pico Western system, Pierce or Western Lightning™ Chemiluminescence Reagent Plus, PerkinElmer).

Immunoprecipitations. Aliquots (100-500 μg) of protein extracts were diluted to 500 μl with dilution buffer and incubated with appropriate antibodies, incubated overnight at 4° C., and mixed with 40 μl of Protein G-Sepharose, Fast Flow® (Amersham Pharmacia Biotech U.K., Ltd., Little Chalfont, Buckinghamshire, U.K.) for one hour. The beads were washed twice in Buffer A, twice with Buffer B, and proteins were eluted in 10-40 μl of loading SDS-PAGE buffer by boiling.

In order to detect small changes in cognitive performance, a particularly sensitive assessment procedure was employed, a procedure capable of measuring transient cognitive changes under the extremely low dose conditions that represent physiological levels of proteins such as oligomeric Aβ. To do this, a procedure originally developed to assess Food Rewarded Behavior (Ettinger and Staddon, Behav Neurosci 97, 639 (1983)) was modified so as to enable the assessment of the cognitive effects of very small doses of psychoactive drugs (Weldon et al., Pharmacol Biochem Behav 54, 753 (1996)). This procedure, the Alternating Lever Cyclic Ratio (ALCR) test, has proven to be more sensitive than many previously published methods for measuring drug effects on cognitive function (Richardson et al., Brain Res 954, 1 (2002), O'Hare et al., Behav Pharmacol 7, 742 (1996)). Briefly, rats must learn a complex sequence of lever-pressing requirements in order to earn food reinforcement in a two-lever experimental chamber. Subjects must alternate between two levers by switching to the other lever after pressing the first lever enough to get food reward. The exact number of presses required for each food reward changes, first increasing from two responses per food pellet up to fifty-six presses per food pellet, then decreasing back to two responses per pellet. Intermediate values are based on the quadratic function, x²−x. One cycle is an entire ascending and descending sequence of these lever press requirements (e.g., 2, 6, 12, 20, 30, 42, 56, 56, 42, 30, 20, 12, 6, and 2 presses per food reward). Six such full cycles are presented during each daily session. Errors are scored when the subject perseveres on a lever after pressing enough to get the food reward, i.e., does not alternate (a perseveration error), or when a subject switches levers before completing the response requirement on that lever (a switching error).

ALCR Development. The ALCR assessment procedure was developed over several years. Several potentially confounding variables that might affect the results in the ALCR task, such as sensitivity, motivation and activity, were systematically assessed in the various published studies listed in Table 1 (IH=intrahippocampal; PO=oral; ICV=intracerebroventricular; FOA=Forced Operant Alternation task; PR=Progressive Ratio task).

TABLE 1

| | | Compounds | | ALCR | ALCR Running | Direct test of |
|---|---|---|---|---|---|---|
| Reference | Assessments | Tested | Route | Cognitive Effect | Response Rate | Motivation |
| Cleary et al.¹ | ALCR | Aβ oligomers | ICV | Increased errors | No effect | |
| Richardson et al.² | ALCR | Aβ | IH | Increased errors | | |
| | ALCR | ibuprofen | PO | Normalized errors | No effect | |
| MacNabb et al.³ | ALCR | methimzole | PO | Slow learning | No effect | No effect |
| | FOA, PR | thyroxine | IP | | | |
| O'Hare et al.⁴ | ALCR | Aβ | IH | Increased errors | No effect | |
| MacNabb et al.⁵ | ALCR | methimzole | PO | Slow learning | No effect | No effect |
| | FOA, PR | | | | | |
| Weldon et al.⁶ | ALCR | atropine | IP | Increased errors | No effect | |
| | | | | | Reduced | |
| | | | | | (1 mg/kg) | |

¹Cleary et al., Nat. Neurosci. 8, 79 (2005).
²Richardson et al., Brain Res. 954, 1 (2002).
³MacNabb et al., Neurosci. Res. 36, 121 (2002).
⁴O'Hare et al., Brain Res. 815, 1, (1999).
⁵MacNabb et al., Brain Res. 847, 231 (1999).
⁶Weldon et al., Pharmacol. Biochem. Behav. 54, 753 (1996).

Sensitivity. There is ample empirical evidence in the literature that the ALCR: 1) is at least as sensitive to pharmacological challenge as many other assays (see, for example, MacNabb et al., Brain Res 847, 231 (1999)); 2) produces reliable (repeatable) results under daily testing for months (see, for example, O'Hare et al., Brain Res 815, 1 (1999)); 3) has external validity through comparison with results from other cognitive or memory assays using well characterized pharmacological agents (see, for example, Weldon et al., Pharmacol Biochem Behav 54, 753 (1996)); 4) has empirical evidence of internal validity (see below for discussion of motivational, motoric, and activity issues); and 5) has previously been shown to be affective in assessing several forms of the Aβ protein (see, for example, Cleary et al., Nat Neurosci 8, 79 (2005) and Richardson et al., Brain Res 954, 1 (2002)).

In terms of the relative sensitivity of ALCR to other useful tests of cognitive function and memory, Weldon and colleagues tested the effects of atropine, a common anti-cholinergic agent known to affect memory and cognitive function (Weldon et al., Pharmacol Biochem Behav 54, 753 (1996)). They found that 1.0 mg/kg atropine IP increases switching errors significantly. The typical IP atropine dose range in many maze studies reported in the literature is from 3 to 55 mg/kg.

Motivation. All non-reflexive behavioral assays are necessarily affected by manipulations of motivation, but concerns may arise about susceptibility of the task to uncontrolled motivational variables, ability of the task to differentiate or discriminate between motivational and cognitive effects, and potential motivational effects of small soluble Aβ oligomers that might be mistaken for cognitive effects.

Uncontrolled motivational variables. In the ALCR task rats respond for food (45 mg pellet) under mild food restriction (90% free feeding weight). Several ways to control variability due to unintended changes in the value of the food reinforcer are employed. Each rat is given an exact amount of (weighed) food, and the amount of food is adjusted daily based upon current weight. Under these circumstances the rat's weight is precisely controlled to within ±2-3% of the target weight prior to each session. On the rare occasions when weight exceeds norms, such as sometimes happens early in training or when rats are not run on weekends, experimental compounds are not injected and those data are not included in baseline calculations. Also, rats are fed their entire daily ration just after finishing the daily behavioral session so as to minimize any food-related variability on the next day's session.

Discrimination between motivational and cognitive effects. A good cognitive assay should demonstrate sensitivity to cognitive change under conditions where motivation is unaffected. Few cognitive/memory assays produce specific empirical evidence on this question in each publication, but some tests, like the Morris Water Maze, possess high face validity on this issue. Because the complexity of the ALCR task precludes overt face validity on this issue, an issue was addressed directly in two previously published reports using the classic motivational assessment assay, the Progressive Ratio (PR) procedure developed by Hodos in 1961 (Hodos, Science 134, 943 (1961)). The PR assay requires an increasing number of lever presses for each successive reinforcer. Eventually the number of presses required for a reinforcer exceeds the value of that reinforcer. At that point the subject stops responding and the number of responses in the last ratio completed is called the ‘break point’ and is considered a measure of reinforcer efficacy or motivation (Hodos and Kalman, J Exp Anal Behav 6, 387 (1963)). Break points under PR schedules have been shown to be sensitive to many typical factors influencing motivational level, such as reinforcer magnitude (Cleary et al., Brain Res 653, 210 (1994)), self-administered drug dose (Griffiths et al., Psychopharmacology (Berl) 65, 125 (1979)), or food deprivation level (Macenski et al., Psychopharmacology (Berl) 112, 204 (1993)). In one study, break points in the PR task and cognitive function in the ALCR task were both assessed in animals treated with methimazole in utero (MacNabb et al., Brain Res 847, 231 (1999)). This treatment results in animals that exhibit congenital hypothyroidism. The hypothyroid rats showed significantly slower learning to criterion than controls in the ALCR task, but their motivation for food was not different from that of controls as measured in the PR task.

In a second study using congenitally hypothyroid rats with replacement thyroxin treatment, all treated groups showed normal learning to criterion and error rates, while untreated groups showed impaired acquisition and increased errors compared to controls (MacNabb et al., Neurosci Res 36, 121 (2000)). When motivation for food was assessed in the PR task, all groups (treated and untreated congenital hypothyroid) performed equally well. These data clearly show that the ALCR is capable of detecting cognitive deficits without changes in motivational status. Potential effects of Aβ on motivation for food. The above research does not preclude a motivational effect of Aβ*56 in the current study. However, no published paper or presentation has proposed that any form of Aβ affects motivation for food. While all behaviours are eventually affected by progressive neurodegenerative conditions like Alzheimer's disease, the present example deals specifically with initial Aβ oligomeric effects early in the disease process, long before the appearance of plaques and neurodegeneration. This issue with the response rate data described is also addressed under the heading “Activity.” In addition, it is noted that animals never left pellets in the food receptacle under any of the drug conditions.

Activity. Drug-induced motoric effects can have profound effects on cognitive assays. This issue in the ALCR task has been addressed in previous published studies (see Table 1). It is worth noting that in the current study data from subjects that did not complete all ratio requirements in each cycle was not included in the analysis. Thus, the number of opportunities to make errors is always the same under baseline and treatment conditions regardless of the rate of response and the number of reinforcers earned is constant across sessions and conditions. Each subject's error data under treatment is always compared to that subject's error data under non-treatment (baseline) conditions in a within-subject design. Different forms of response rate data are addressed below.

Absolute responses are equal to the sum of the products of each ratio value RV (2, 6, . . . 56) times its occurrence, plus total errors (perseveration plus switching errors). This is not a useful assessment of activity since compounds that increase errors will always have higher absolute responses.

Absolute response rates (or total responses per session length) is a better measure, but post reinforcement pause contributes significantly to session length. As demonstrated in several other published studies, drugs that increase lever choice errors often disrupt the subject's ability to keep track of ratio size, resulting in a flattened slope for the PRP by Ratio Value function (see, for example, O'Hare et al., Behav Pharmacol 7, 742 (1996)). A flattened PRP function decreases session length while the associated increased errors drive total responses up. Under these circumstances absolute response rate is a less than perfect metric of activity or motivation. Nevertheless, interpretation of ALCR error rates is stronger if absolute response rates (correct+incorrect responses divided by session length) are stable. To evaluate this effect, absolute response rates after infusion of Aβ*56 and control injectates were calculated (FIG. 11). No statistically significant differences in absolute response rate were found between these conditions.

Running Response Rate. The best indicator of changes in activity level, potential drug-induced motoric impairment, and motivation under this lever pressing task is the Running Response Rate (RRR). RRR measures the responses per second during the time the animal is actually responding on the lever. RRR, of course, has a direct relationship to the size of the ratio requirement. This measure is easily affected by sedating drugs and drugs that deleteriously affect motor function. FIG. 12 shows RRR plotted across all ratio values for the current study. There were no statistically significant differences in RRR between any of the injection groups. Differences were tested using multiple RMANOVAs, at each ratio value and no significant differences in RRR were found across treatments.

Thus, no differences in response rates in either absolute (total) responses per minute or running response rates were found under treatment conditions that produced significant changes in errors. To the extent that activity and motivation are reflected in these response rates, Aβ*56-induced errors in the ALCR task are not confounded by changes in activity or motivation.

ALCR Cognitive Effects

Prefrontal Executive Function. It is likely the ALCR task can measure cognitive changes involving prefrontal executive function. In fact, empirical evidence of this effect was obtained using hypothyroid rats in the ALCR task. The results in the ALCR task relate to results seen in hypothyroid humans and monkeys (MacNabb et al., Brain Res 847, 231(1999), MacNabb et al., Neurosci Res 36, 121 (2000)). Frontal lobe executive function affecting choice behavior in humans is often assessed by the Wisconsin Card Sorting Test, wherein subjects are rewarded for choosing cards based upon stimulus class or set (e.g., shape) and a rule relating classes (e.g., square then green). A key feature is that the rule changes during the task. Humans with frontal lobe damage typically perseverate and do not apply the new rule. The present data on increased perseveration errors in the ALCR task are consistent with this general paradigm of cognitive dysfunction. A discussion of these effects can be found in MacNabb et al., 1999 (MacNabb et al., Brain Res 847, 231(1999)).

Hippocampus Involvement. Previously published research supports ALCR's sensitivity to direct disruption of hippocampal systems. In one study, Aβ was aggregated prior to injection at 10⁻⁴ M and injected bilaterally into rat hippocampal formations (O'Hare et al., Behav Pharmacol 7, 742 (1996)). Perseveration errors, i.e., errors resulting from the rat choosing the incorrect lever after the Post Reinforcement Pause (PRP), increased significantly after IH Aβ injection. The PRP is discussed more fully below. Histological examination of the brains ninety days post injection showed accurate injectate placement and revealed that aggregated Aβ was still present in the hippocampus. A recent study used ALCR to assess the effects of a non-steroidal anti-inflammatory drug, ibuprofen, on Aβ-induced deficits following IH injection of a suspension of pre-aggregated Aβ (Richardson et al., Brain Res 954, 1(2002)). Results from this study replicated the findings of O'Hare et. al., 1999 (see O'Hare et al., Brain Res 815, 1(1999)) in regard to A13-induced ALCR performance decrements and also demonstrated that ibuprofen mitigated these effects. These studies clearly show the ALCR is sensitive to direct hippocampal disruption.

Memory. ALCR was not specifically designed as a test of memory, however, some form(s) of memory are involved in this or any task that requires the animal to learn and perform—such as learning to press a lever to earn food. An additional form of memory is involved in discriminating between response options or choosing (e.g., switching rules), dependent upon the past occurrence of some event (e.g., food delivery). These memory forms are typically captured under the general rubric of ‘reference’ memory, for memory of the ‘rules/requirements of the task.’ Whether ALCR was sensitive to this type of memory disruption was specifically addressed by comparing choice responding in the ALCR, Forced ALCR and Forced Operant Alternation (FAO) tasks with multiple fixed-ratio requirements (MacNabb et al., Brain Res 847, 231 (1999), MacNabb et al., Neurosci Res 36, 121 (2000)). In the FOA and Forced ALCR tasks, lever pressing still alternates but there is only one choice available after the PRP. In this study, only the ALCR task differentiated between hypothyroid and normal animals in their ability to reach a learning and accuracy proficiency criterion. Hypothyroid rats did not differ in reaching the learning criterion in the Forced ALCR or simple operant Forced Alternation tasks with multiple fixed ratio values, reflecting the added value of learning the switching rules.

To claim that ALCR incorporates a working memory component there must be a delay between the response choice and the absent temporally discrete discriminative stimulus upon which it is based. Usually, these delays are independent variables imposed by the experimental procedure. In the ALCR task there is a reliable delay between discriminative stimulus and response but it is a dependent variable, i.e., under the control of the subject. The delay, called a Post Reinforcement Pause (PRP), has been shown to occur reliably after completing a ratio response requirement, like those in the ALCR procedure. The PRP reliably occurs in many species and under a great diversity of conditions, from birds pecking lighted disks to humans playing slot machines (Schreiber and Dixon, Psychol Rep 89, 67 (2001)). The PRP is directly related to the size of the ratio requirement. As can be seen in FIG. 13, mean PRPs in the ALCR task are directly related to ratio size. It should be clear from FIG. 13 that PRP is not feeding behavior but is, as consistently reported in the operant literature, a direct function of ratio size. It should also be noted that mean PRPs after infusions of Aβ*56 are reduced relative to baseline under the higher ratio sizes. Even though this reduction is not statistically significant, it would if anything make the correct lever choice after infusions of Aβ oligomers easier and thus work against a significant finding of increased perseveration errors reported in the present example. It is indicative of effects seen with several drugs known to affect cognitive function in the ALCR task and reflects the subjects' failure to track the size of the current ratio size. While the data in FIG. 13 suggests that rats given Aβ*56 were impaired in their ability to track the ratio requirement (see O'Hare et al., Behav Pharmacol 7, 742 (1996)), the effects did not reach statistical significance. In summary, there is a consistent pause after completing a response requirement and before the choice that might result in a perseveration error in the ALCR task. However, despite the contention that there is a consistent delay prior to the choice response, it is acknowledged that the situation in the ALCR task does not mimic typical procedures used to measure working memory.

TABLE 2
Levels of various Aβ species in Tg2576 mice.

| | | Ages Tested | Appearance | |
|---|---|---|---|---|
| Type of Aβ | Citation | (months) | at 6 months | Fold-change in Aβ± |
| ADDLs | Chang et al.¹ | 13, 17 | ND | ˜5x in septum, ˜20-100x |
| | | | | in cortex |
| Aβ42_{SDS} | Kawarabayashi et al.² | 2-23 | no | ˜7-8x |
| Aβ42_{FA} | Kawarabayashi et al.² | 2-23 | yes | ˜500-1000x |
| Aβ42_{SDS} | Kawarabayashi et al.² | 2-23 | no | ˜10x |
| Aβ42_{FA} | Kawarabayashi et al.² | 2-23 | yes | ˜1000x |
| Lipid raft Aβ | Kawarabayashi et al.³ | 4-12, 17, 24 | yes | >500x |
| Intracellular Aβ | Takahashi et al.⁴ | 3, 10 | no | ˜2x |

‡Fold-change in Aβ between 6-13 months, except for ADDLs and intracellular Aβ, where fold-change is denoted for the ages tested.
Abbreviations:
ND, not determined;
ADDLs: Aβ-derived diffusible ligands;
AβSDS: SDS-extractable Aβ;
AβFA: SDSinsoluble, formic acid-extractable Aβ.

Three Aβ species first appear at six months: 1) SDS-insoluble Aβ40; 2) SDS-insoluble Aβ42; and 3) Aβ in lipid rafts (Kawarabayashi et al., J Neurosci 21, 372 (2001); and Kawarabayashi et al., J Neurosci 24, 3801 (2004)). However, all three Aβ species increase 500- to 1000-fold in the interval from six to fourteen months of age when there is little or no further change in memory. Other Aβ species, including intracellular Aβ (Takahashi et al., J Neurosci 24, 3592 (2004)) and SDS-soluble Aβ (Kawarabayashi et al., J Neurosci 21, 372 (2001)) are already present prior to 6 months of age, and therefore are probably not involved in memory loss occurring at six months. ADDL levels increase 20- to 100-fold in the cortex between 13 and 17 months of age (Chang et al., J Mol Neurosci 20, 305 (2003)), and 130-fold in the hippocampus between nine and twenty months of age.

In all figures, the ages of mice (in months) are indicated above each gel in bold characters below the corresponding genotype. Tg2576−/−, Tg2576+/−, and Tg2576+/+ denote mice harboring zero (non-Tg), one and two transgene arrays, respectively. Numerals inside bars denote numbers of mice. Spatial memory refers to the retention of spatial information evaluated in the Morris water maze as measured by mean % target quadrant occupancy scores±S.E.M during probe trials. When indicated, synthetic human Aβ₁₋₄₂ peptide (hAβ₄₂) was loaded in parallel as a size marker and positive control (right lane). Arrows indicate respective migration positions of monomers (1-mer), dimers (2-mer), trimers (3-mer), tetramers (4-mer), hexamers (6-mer), nonamers (9-mer) and dodecamers (12-mer), as well as sAPPα. Western blot (WB), immunoprecipitation (IP).

FIG. 1 shows temporal patterns of memory decline and soluble, extracellular Aβ oligomers in Tg2576 mice. The spatial memory in mice from 4 to 17 months shows a progressive but irregular decline with periods of stability (FIG. 1A). Temporal analysis of spatial memory shows three stages of performance (FIG. 1B). In FIG. 1C the identification of Aβ oligomers in soluble, extracellular-enriched extracts of proteins from brains of 5-, 6-, and 7-month mice, was assessed by western blot (WB) with or without immunoprecipitation (IP). The intensity of the 40 kDa band co-migrating with nonamers was 33.92±12.5% (n=4) of the intensity of the 56 kDa band corresponding to dodecamers.

FIG. 2 shows that water maze performance correlates inversely with Aβ nonamer and dodecamer levels. There is a lack of significant correlations between spatial memory and monomeric, trimeric or hexameric soluble AO species detected in the extracellular-enriched fractions of 5-month and 6-month Tg2576+/− mice (FIG. 2A-2F). FIGS. 2G and 2H show that the nonameric and dodecameric Aβ levels correlate inversely with spatial memory at 6 months.

As shown in FIG. 3, Aβ*56 disrupts cognitive function. Soluble proteins in extracellular-enriched extracts from Tg2576+/− or Tg2576−/− mice were fractionated by SEC to generate fractions with (19+/−) or without (17+/− and 19−/−) the 56 kDa Aβ species (FIG. 3A). Fraction 19+/− was also subjected to three rounds of IP with 6E10 to remove the 56 kDa Aβ species. Bands at 75 and 150 kDa present in non-Tg littermates and IP19+/− were considered to be non-specific (ns). Perseveration Errors (FIG. 3B) and Switching Errors (FIG. 3C), presented as a percentage of baseline rates (±SEM), in the ALCR paradigm two hours after rats received injections of the indicated fractions into the lateral ventricles. Both types of errors increased significantly above baseline only when the 56 kDa Aβ species was present. (Wilcoxon matched-pairs signed-ranks test, *p<0.05).

FIG. 4 shows that the elimination of Aβ*56 coincides with an interlude of normal spatial memory. Aβ*56 levels decline in Tg2576+/− mice between 12.0-12.4 months of age (FIG. 4A). Levels of Aβ*56 between 10.7-13.0 months of age (FIG. 4B). Values represent band intensities (mean ±SD) relative to the intensities observed at 10.7 months. ANOVA, p<0.02, followed by t test, *p<0.01, n=4 animals per age group. The dip in Aβ*56 levels coincides with a transient recovery of spatial memory (target quadrant occupancy SEM in Day 5 probe trial) (FIG. 4C). ANOVA, p<0.01, followed by t test, *p<0.01, **p<0.001, and by Fisher's protected least significant difference (PLSD) test, ‡p<0.001, #p<0.05. Tg2576+/−, Tg2576−/−. FIG. 4D shows kinetics of the rate of change in Aβ(x-42) levels (SDS-soluble Aβ(x-42) and SDS-insoluble Aβ(x-42) in Tg2576+/− mice (adapted from Kawarabayashi et al., 2001(11)). Abbreviations: FA=SDS-insoluble/formic acid-soluble; Δt=time interval. And, FIG. 4E shows a hypothetical dynamic relationship between soluble and insoluble pools of Aβ.

FIG. 5 shows the fidelity of the technique for measuring soluble, extracellular Aβ oligomers in vivo. In FIG. 5A procedures used to extract various pools of Aβ are illustrated. FIG. 5B shows an SDSPAGE analysis of several protein markers in the collected fractions. The forebrain was subjected to a four step extraction protocol generating four fractions (extracellular-enriched soluble, intracellular-enriched soluble, membrane-enriched, and insoluble). Partial characterization and validation of the fractionation procedure was achieved using the NMDA receptor subunit NR2 as a marker for membrane-enriched proteins, APP as a marker for both soluble (sAPPα) and membrane-enriched (full-length APP=fl-APP) proteins, and the extracellular serine protease tissue-type plasminogen activator t-PA, as a marker for soluble proteins. Other protein markers used were microtubule-associated proteins MAP-2 and tau (cytoskeleton), the protein kinases, ERKs and JNK (cytosol), and flotillin-2 (lipid rafts). Soluble microtubule-free tau, ERKs, JNK and c-Jun were mainly identified in the intracellular-enriched soluble fraction; cytoskeleton proteins MAP-2 and microtubule-bound tau were present within membrane-enriched fractions; and flotillin-2 was found in the insoluble pellet resuspended with Tris-buffered formic acid. FIG. 5C presents validation of the use of immunoprecipitation to study Aβ species quantitatively. To ensure that immunoprecipitated Aβ species were a faithful index of brain Aβ levels, two sequential immunoprecipitations (IP1 and IP2) with either 6E10 or 4G8 monoclonal antibodies were performed and revealed negligible amounts (1±0.64%, n=4) of 6E10 immunoreactive material in WB after the first IP in extracts from the oldest animals (25 months).

FIG. 6 presents the biochemical and structural properties of Aβ assemblies in Tg2576 mice. FIG. 6A demonstrates the purification of soluble, extracellular-enriched Aβ species using affinity columns packed with 200 μg of 6E10 or 4G8 antibodies. Captured proteins were eluted in acidic buffer (pH 3), fractionated by SDS-PAGE, and WB were revealed with 6E10. FIG. 6B shows that Aβ multimers are resistant to the strong chaotropic agent, 8M urea. Soluble, extracellular-enriched extracts from 12- to 20-month Tg2576+/− brains were loaded onto 8M urea containing SDS-PAGE gels, electrotransfered, and probed with 6E10. The presence of urea did not alter electrophoretic migration patterns, indicating that Aβ oligomers are probably not associated with large globular proteins. FIG. 6C demonstrates that soluble HMW Aβ oligomers are not resistant to treatment with greater than or equal to 15% hexafluoroisopropanol (HFIP). Monomeric Aβ levels increased with rising HFIP concentrations. Trimers were exceptionally resistant to HFIP. FIG. 6D represents an evaluation of Aβ multimers with the anti-oligomer antibody, A11. Note that hAs42 standards are not detected with the A11 antibody (right lane).

FIG. 7 is a characterization of native Aβ oligomer size and expression levels in Tg2576 mice. FIG. 7A shows a SDS-PAGE analysis of soluble brain extracts fractionated by SDS-free size exclusion chromatography (SEC) showed that all Aβ oligomers migrated at expected molecular weights using globular protein standards. These data confirm that high molecular weight (HMW) Aβ oligomers are not artificially generated during electrophoresis from monomeric or trimeric Aβ species, and trimers are not degradation products of HMW oligomers. Bands revealed at 75 and 150 kDa are non-specific bands which are also present in blots of extracts from non-transgenic mice. In FIG. 7B soluble, extracellular-enriched Aβ species are assessed by WB using 6E10 in mice between 9 and 25 months of age. FIGS. 7C and 7D represent a semi-quantification of Aβ species levels (relative to β-tubulin levels) expressed as percentage of respective averaged signals observed in 9-month-old animals (n=6/age group). ANOVA, followed by t test, *p<0.01, #p<0.05.

FIG. 8 shows the absence of a correlation between Aβ oligomer levels and swimming speed or path length during the cued (or visible) phase of water maze testing. FIGS. 8A and 8B show the relationship between swimming speed and Aβ levels in 5-(FIG. 8A) and 6-(FIG. 8B) month-old Tg2576+/− mice. ANOVA p values are displayed in graphs alongside r2 values. FIG. 9 shows there is no change in levels of intracellular Aβ levels and in CTFs in 5- and 6-month Tg2576 mice. In FIG. 9A soluble, intracellular-enriched Aβ species in 5-, 6- and 7-month mice evaluated by western blot (WB) using 6E10. The top insert shows levels of sAPPα. FIG. 9B demonstrates compartmentalization of APP-CTFs in Tg2576 mouse brain. CTF-β's were IP'd from soluble or membrane extracts with 6E10 and identified in WB with APPCter-C17. No immunoreactive bands were detected in the soluble fraction, but a doublet of bands around approximately 2 kilodalton (kDa) was present in the membrane-enriched fraction. As a control, both CTF-β and CTF-α were detected with APPCter-C17. FIG. 9C presents IPs of CTFs using APPCter-C17 confirmed no overall change in CTFs between 5 to 7 months of age. Full-length APP also captured by the antiserum is displayed on top and shows no variation in levels. Trimers are the predominant oligomeric Aβ species secreted from Tg2576 cultured primary brain cells. Near pure primary cortical neurons or neurons cocultured with astrocytes immunolabelled with antibodies for MAP-2, GFAP and DNA intercalants DAPI or Propidium Iodine (Ipr). In FIG. 10A the levels of naturally secreted Aβ species in the conditioned media (CM) of 7- or 14-DIV (days in vitro)-old neurons were evaluated by IP followed by WB with 6E10 antibodies. In CM from 7-DIV neurons trimers are most prominent. In CM from 14-DIV neurons tetramers and trimers are present, but monomers are barely detectable (without boiling the membrane). FIG. 10B shows that cortical astrocytes modulate the levels of neuron-derived Aβ species in the CM. When Tg2576+/+ neurons were co-cultured in the presence of Tg2576−/− astrocytes, the overall levels of Aβ diminished and trimers were the only species detected in the CM (without boiling the membrane). FIG. 10C shows that boiling the membranes enhances the detection of monomeric Aβ with 6E10, which constitutes the major Aβ species secreted in the CM. However, boiling did not significantly enhance the ability to detect the oligomeric Aβ species, presumably because the tertiary and quaternary structure of the trimers and tetramers readily exposes the 6E10 epitope without requiring further denaturation. FIG. 10D intracellular protein preparations from Tg2576+/− primary neurons devoid of APP-CTFs were IP'd with 6E10, revealing trimers but not tetramers, just as in the soluble, intracellular-enriched fractions of 5- to 6-month Tg2576+/− mice (see FIG. 9A). These data suggest that trimers are formed within neurons and subsequently secreted. HMW Aβ oligomers are assembled outside of neurons. FIG. 10E demonstrates membrane-associated APP-derived molecules in Tg2576+/− primary neurons. IPs using 6E10 captures CTF-βs and Aα monomers. The blot was denatured and re-probed with APPCter-C17 to confirm the nature of the approximately 13 kDa bands, revealing both phosphorylated (pCTF) and nonphosphorylated CTF-β. When extracts were immunodepleted of APP-CTFs, only Aβ monomers remained.

FIG. 11 presents overall response rates. Response rates of rats were compared at Baseline and after receiving fractions from Tg Pos Lane 19 (Lane 19+/− in FIG. 3) containing Aβ*56 from a Tg2576 transgene positive mouse, corresponding Tg Neg Lane 19 (Lane 19−/− in FIG. 3) from a transgene negative mouse, and Tg Control Lane 17 (Lane 17+/− in FIG. 3) without Aβ*56 from a transgene positive mouse. There were no significant differences between the overall response rates.

FIG. 12 presents running response rates. Running response rates of rats were compared at Baseline and after receiving fractions containing Aβ*56 from a Tg2576 transgene positive mouse Lane 19+/− in FIG. 3) and Tg2576 Control Ln 17 (Lane 17+/− in FIG. 3) without Aβ*56 from a transgene positive mouse. There were no significant differences between the running response rates.

FIG. 13 presents post reinforcement pause as a function of ratio size. Post reinforcement pause was compared as a function of ratio size in rats at Baseline and after receiving fractions containing Aβ*56 from a Tg2576 transgene positive mouse (Lane 19+/− in FIG. 3) and Tg2576 Control Ln 17 (Lane 17+/− in FIG. 3) without Aβ*56 from a transgene positive mouse. There were no significant differences between these values.

### Example 2

### The Isolation of Aβ* from B6SJL and 129FVBF1 Mice

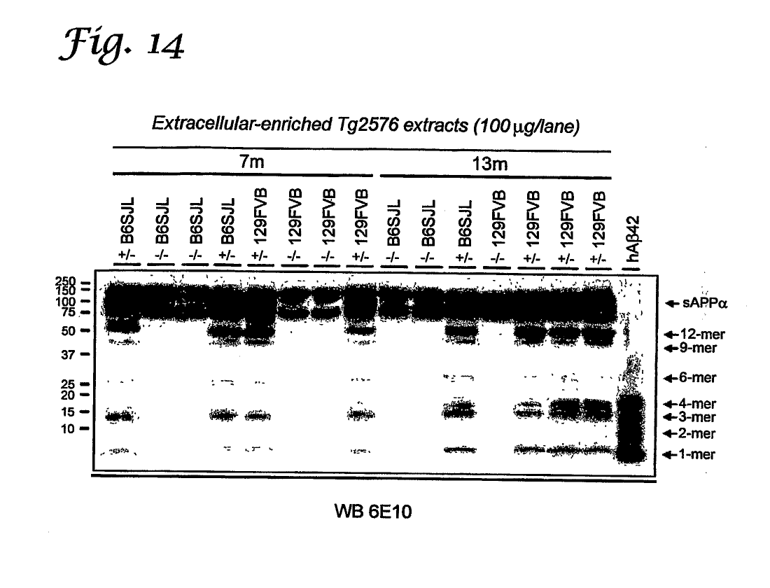

Following the procedures detailed in Example 1, FIG. 14 demonstrates that B6SJL and 129FVBF1 mice show identical patterns of soluble Aβ oligomers at various ages. This argues against the potential effects of strain background on Aβ formation.

### Example 3

### Antibodies to Aβ*

Binding Specificities and Screening Methodology for Antibodies Directed Against Aβ*

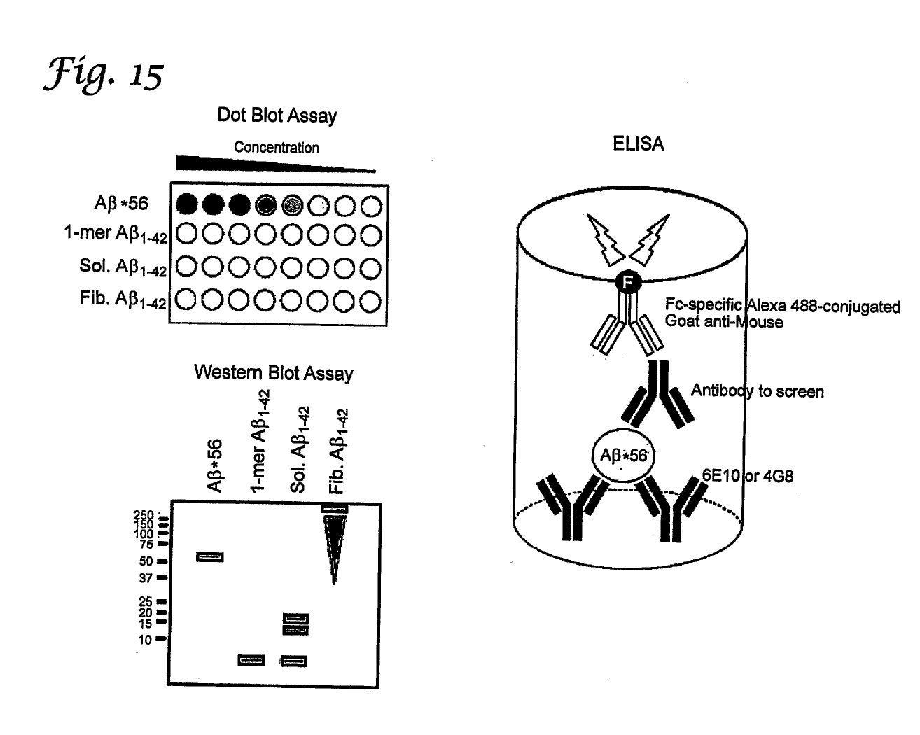

Candidate anti-Aβ* clones will be screened comprehensively using methods that ensure that the anti-Aβ* monoclonals specifically recognize natively folded Aβ*56, and do not bind fibrillar or monomeric Aβ. Dot blot methods followed by confirmatory liquid-phase immunoprecipitation and immunoblotting experiments will be used for this purpose. Direct liquid phase ELISA methods will also be used. These dual methods are depicted in FIG. 15. The dot blot method is advantageous due to its rapid throughput and minimal potential for steric hindrance preventing detection of suitable clones. The ELISA method is useful due to its ability to detect natively folded Aβ*56 directly.

FIG. 15 shows various methods for screening candidate monoclonals for antibodies that specifically detect Aβ*56. In the Dot Blot assay, Aβ*56, synthetic monomeric Aβ(1-42), soluble Aβ(1-42) oligomers and fibrillar Aβ(1-42) will be spotted at known concentrations on nitrocellulose or nylon filters. The filters will be overlaid with candidate monoclonals. Clones that selectively stain A13456 at low concentrations will be selected. In the Western Blot assay, which is a confirmatory test for the Dot Blot assay, Aβ*56, synthetic monomeric Aβ(1-42), soluble Aβ(1-42) oligomers and fibrillar Aβ(1-42) will be size-fractionated by polyacrylamide gel electrophoresis and transferred to nitrocellulose or nylon filters. The filters will be overlaid with candidate monoclonals. Clones that selectively stain Aβ*56 but no other forms of Aβ will be selected. In the liquid phase ELISA method, monoclonal anti-Aβ antibodies 6E10 or 4G8 will be immobilized onto the wells of plastic plates, overlaid with Aβ*56. Candidate monoclonals will be applied to wells. Clones that bind Aβ*156 will be detected with goat anti-mouse antibodies conjugated to a fluorescent marker.

Making Antibodies Directed Against Aβ*

To generate anti-Aβ* monoclonals, mice will be immunized with purified Aβ*56 from the brains of Tg2576 mice greater than six months old, AD patients, or Down syndrome patients, or with synthetic Aβ oligomers that include species which are 56 kDa. Aβ*56 will be purified by immunoaffinity chromatography followed by size-exclusion chromatography so that it runs as a single band on silver stained gels, as we have previously shown. Biochemical methods will also be used to purify Aβ*56, taking advantage of the stability of Aβ*56 in 8M urea, which denatures most globular proteins.

To determine that the purified immunogen is biologically active, it will be assayed for its ability to inhibit NMDA-evoked currents in cultured neurons, prior to injection as an immunogen. However, this is not an essential step, because the screening method described above will select only those monoclonals that specifically detect Aβ*56.

It is expected that these methods will successfully lead to the generation of specific antibodies to Aβ*56, particularly since multimerized proteins tend to be better immunogens than monomeric proteins, because they crosslink immunoglobulins on B-cells.

A source of concern is that the use of an immunogen consisting of 56 kDa Aβ oligomers generated from synthetic Aβ would theoretically yield fewer monoclonals that target the specific conformation of natively folded Aβ*56. For this reason, the screening of candidate anti-Aβ* monoclonals using natively folded Aβ*56, which is shown in FIG. 15, is important.

It is possible that Aβ*56 in human brain (from AD patients) and mouse brain (from Tg2576 mice) may differ subtly in conformation. Therefore, both natively folded Aβ*56 purified from AD and Tg2576 mouse brain tissue will be used to screen monoclonals. The results will generate a two by two catalogue of clones showing specific recognition of AD-Aβ*56, Tg2576-Aβ*56, both AD-Aβ*56 and Tg2576-Aβ*56, or neither protein complex. This catalogue will aid in selecting the most appropriate anti-Aβ* monoclonals for use in humans.

Assessing Therapeutic Efficacy of Anti-Aβ* Monoclonals in Behavioral and Electrophysiological Assays

Three approaches will be used to assess the functional efficacy of anti-Aβ* monoclonals, which have been biochemically validated using the methods described above. Additional behavioral and electrophysiological assays will be used to determine whether the anti-Aβ* monoclonals are functionally effective in neutralizing the detrimental effects of Aβ*. Monoclonals with the highest binding affinities to purified, natively folded Aβ*56 will be used for functional assays.

In the first approach, intraperitoneally (IP) administered anti-Aβ* monoclonals will be assessed for their ability to prevent and to reverse spatial reference memory deficits in Tg2576 mice. In the second approach, anti-Aβ* monoclonals administered IP or injected directly into the lateral ventricles will be evaluated for their ability to block the disruption of cognitive function in healthy rats receiving Aβ*56. In the third approach, the response of Aβ*56 inhibition of NMDA-evoked currents to anti-Aβ* monoclonals will be studied. Monoclonals that show both selective biochemical binding to Aβ*56 and neutralizing effects on the deleterious functional actions of Aβ*56 will be important therapeutic and diagnostic tools.

Assessing Ability of Anti-Aβ*Monoclonals to Ameliorate Spatial Reference Memory Deficits in Tg2576 Mice

To test the effects of IP administered anti-Aβ* monoclonals on spatial reference memory deficits in Tg2576 mice, a protocol previously employed to show reversal of pre-existing memory deficits in Tg2576 following IP administration of BAM-10 (Kotilinek et al., J. Neurosci. 22(15):6331-5 (2002)), a monoclonal antibody raised against Aβ(1-10) which clears plaques in vivo (Bacskai et al., J. Neurosci. 22(18):7873-8 (2002) and Lombardo et al., J. Neurosci. 26; 23(34):10879-83 (2003)) and detects soluble Aβ monomers and oligomers as well as Aβ*56, will be employed. In addition, anti-Aβ* monoclonals will be administered to young, unimpaired mice to assess the prophylactic potential of the anti-Aβ* monoclonals. BAM-10 will serve as a positive control and non-specific immunoglobulin G will serve as a negative control. These experiments will determine the ability of the anti-Aβ* monoclonals to cross the blood brain barrier and to neutralize Aβ*56 or to act as “peripheral sinks” to extract Aβ*56 out of the brain.

Assessing the Ability of Anti-Aβ*Monoclonals to Block Aβ*-Induced Behavioral Deficits in Healthy Rats

The ALCR behavioral protocol (Cleary et al., Nat. Neuroscience 8, 79-84 (2005) and PCT/US2005/023070, filed Jun. 30, 2005) may be used to assay Aβ*56 directly, and also to test the effects of anti-Aβ* monoclonals on AD-Aβ*56 mediated disruption of cognitive function in healthy rats. Anti-Aβ* monoclonals will be administered directly into the lateral ventricles, prior to injecting Tg2576-Aβ*56 or AD-Aβ*56. These experiments will show that the monoclonals specifically inhibit the deleterious effects of Tg2576-Aβ*56 or AD-Aβ*56 on cognitive function.

Aβ* specifically disrupts cognitive function and antibodies directed against Aβ* specifically target a this key causing cognitive deficits. Thus, antibodies directed against Aβ* will be therapeutically effective in ameliorating cognitive deficits. The effectiveness of such administration of antibodies is supported by the prior success of passive and active vaccine studies in mice in preventing and reversing memory loss and the encouraging preliminary reports with immunization with AN-1792, (beta amyloid peptide 1-42) on cognitive function in a subset of Alzheimer's disease (AD) patients (see, for example, Gilman et al., Neurology 64:1553-1562 (2005). The passive administration of such antibodies would avoid the risk of encephalitis caused by a cellular immune reaction associated with active immunization, such immunization with AN-1792.

Aβ* may be present at low levels in serum or CSF in the pre-clinical or very early stages of Alzheimer's disease. Thus, then Alzheimer's disease may be diagnosed by detecting Aβ* in serum or CSF, using antibodies specifically directed against it. For example, with highly specific polyclonal and monoclonal antibodies against Aβ*, nanotechnology may be used to detect quantities of Aβ* in the attomolar (10⁻¹⁸ M) range (see, for example, Georganopoulouet al., Proc Natl Acad Sci USA. 2005 Feb. 15; 102(7):2263-4). Thus, antibodies directed against Aβ* may be used in methods for the early diagnosis or prediction of cognitive disorders, including, but not limited to, AD.

### Example 4

### Human Aβ*56

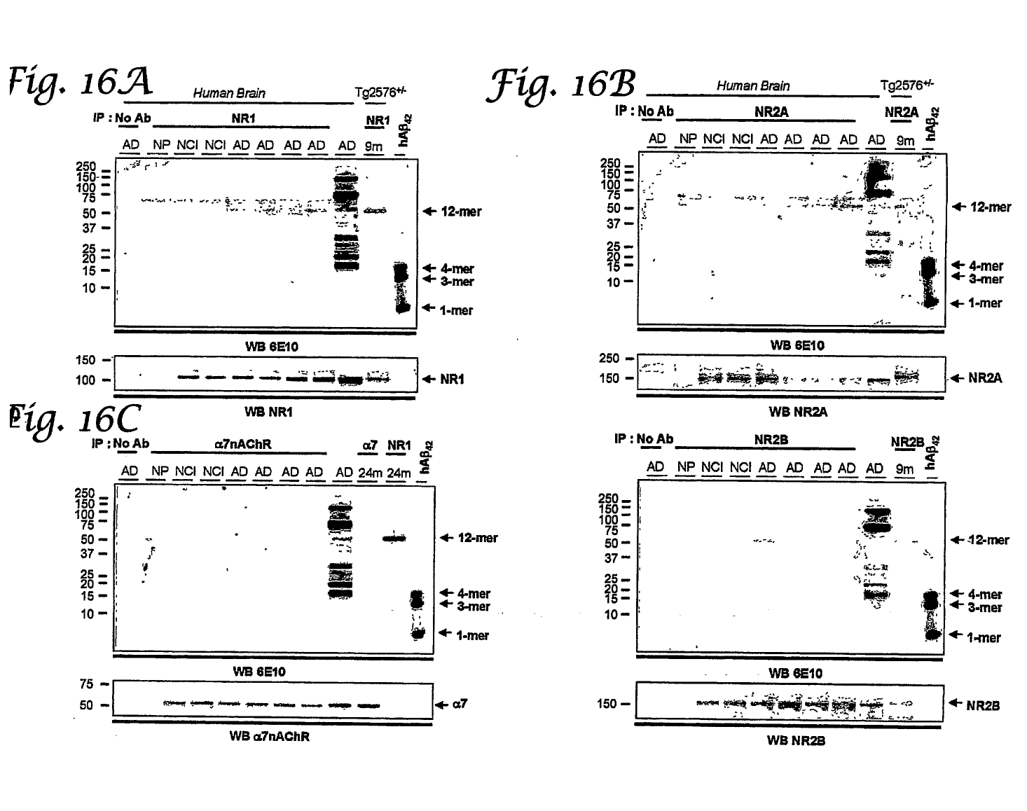

It has been shown that Aβ*56 is a ligand of the NMDA receptors (U.S. Provisional Application 60/703,653, filed Jul. 29, 2005). The interaction of Aβ assemblies and ionotropic glutamate receptors in brain tissue from patients with AD and control individuals without dementia was examined. NR1, NR2A and, to a significantly lesser extent, NR2B antibodies immunoprecipitated a 56-kD 6E10-immunoreactive protein co-migrating with Aβ*56 in brain tissue samples from all four patients with AD, but in neither of two samples from control individuals with no cognitive impairment (NCI) (FIGS. 16A, 16B). The unequal levels of NR2A subunits were not due to inconsistencies in loading samples, and therefore reflected actual receptor subunit levels in the brain specimens. These data indicate that Aβ*56 or an Aβ*56-like molecule binds NMDA receptors selectively in AD patients. Since NR2B subunits are preferentially found in extra synaptic NMDA receptor complexes (Collingridge et al, Wang, Nat Rev Neurosci 5, 952 (2004)), it is possible that in AD brain, Aβ*56 binding of NMDA receptors is biased toward synaptic NMDA receptors. Larger sample sizes will be required to ascertain whether the binding of Aβ*56 or an Aβ*56-like molecule in human brain tissue to NMDA receptors can be used to define AD biochemically.

As shown in FIG. 16. human-derived Aβ*56 physically binds NMDA receptors. FIG. 16A demonstrates that Aβ*56 coimmunoprecipitates with NR1 NMDA receptor subunits in brain tissue from Alzheimer (AD) patients but not from control subjects with no cognitive impairment (NCI), or extracts containing no brain proteins (NP). FIG. 16B demonstrates that Aβ*56 co-immunoprecipitates with NR2A, but much less readily with NR2B, NMDA receptor subunits in brain tissue from subjects with AD but not from control subjects (NCI). FIG. 16C demonstrates that Aβ*56 does not co-immunoprecipitate with α7 nicotinic acetylcholine receptors (α7nAChR). Panels below each blot confirm the ability of the various receptor antibodies to immunoprecipitate the respective receptors or receptor subunits.

Methods

Human brain tissue. Frozen specimens of cerebral cortex were obtained from three AD patients and two cognitively intact control subjects, and one AD patient.

Antibodies. The following primary antibodies were used: 6E10 [1:100-10,000 dilution] against Aβ1-17 (Signet Laboratories, USA) and antibodies raised against NR1 and NR2 subunits (A-D) [1:200 dilution] (Santa Cruz Biotechnologies Inc, USA).

Protein extractions. Soluble, extracellular-enriched fractions were generated from hemi-forebrains harvested in 500 μl of solution containing 50 mM Tris-HCl (pH 7.6), 0.01% NP-40, 150 mM NaCl, 2 mM EDTA, 0.1% SDS, 1 mM phenylmethylsulfonyl fluoride (PMSF), and protease inhibitor cocktail (Sigma). Soluble, extracellular-enriched proteins were collected from mechanically homogenized lysates (1 ml syringe, gauge 20 needle [10 repeats]) following centrifugation for 5 minutes at 3,000 rpm.

Membrane-enriched fractions were generated from hemi-forebrains harvested in 500 μl of solution containing 50 mM Tris-HCl (pH 7.6), 0.1% NP-40, 150 mM NaCl, 2 mM EDTA, 1% SDS, 1 mM PMSF, 2 mM 1,10-PTH and protease inhibitor cocktail (Sigma). Lysates were mechanically homogenized (1 ml syringe and needle, gauge 20 [10 repeats]) and centrifuged for 90 minutes at 13,000 rpm. Membrane-associated proteins were generated from the pellets re-suspended with 500 μl of buffer (50 mM Tris-HCl [pH 7.4], 150 mM NaCl, 0.5% Triton X-100, 1 mM EGTA, 3% SDS, 1% deoxycholate, 1 mM of PMSF) following centrifugation for 90 minutes at 13,000 rpm. All supernatants were clarified by centrifuging for 90 minutes at 13,000 rpm prior to western blot analysis. Protein amounts were determined (BCA Protein Assay, Pierce). Western blot and immunoprecipitations were performed as described in Example 1.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1. An isolated, soluble amyloid-β protein assembly comprising more than one detergent stable oligomer of amyloid-β proteins.

2. The isolated, soluble amyloid-β protein assembly of claim 1 wherein the amyloid-β protein assembly disrupts cognitive function.

3. The amyloid-β protein assembly of claim 1 wherein the amyloid-β protein assembly has a molecular weight of about 40 kDa as measured by SDS polyacrylamide gel electrophoresis.

4. The amyloid-β protein assembly of claim 1 wherein the amyloid-β protein assembly has a molecular weight of about 56 kDa as measured by SDS polyacrylamide gel electrophoresis.

5. The isolated, soluble amyloid-β protein assembly of claim 1 wherein the amyloid-β protein assembly comprises a dodecamer of amyloid-β proteins.

6. (canceled)

7. The amyloid-β protein assembly of claim 5 wherein the dodecamer of amyloid-β proteins comprises four detergent stable trimers of amyloid-β protein.

8. The amyloid-β protein assembly of claim 5 wherein the dodecamer of amyloid-β proteins comprises three detergent stable tetramers of amyloid-β protein.

9-10. (canceled)

11. The isolated, soluble amyloid-β protein assembly of claim 1 comprising more than one detergent stable trimer of amyloid-β proteins.

12. (canceled)

13. The amyloid-β protein assembly of claim 11 comprising four detergent stable amyloid-β protein trimers.

14-15. (canceled)

16. The isolated, soluble amyloid-β protein assembly of claim 1 comprising more than one detergent stable tetramer of amyloid-β proteins.

17. The amyloid-β protein assembly of claim 16 comprising three detergent stable amyloid-β protein tetramers.

18. (canceled)

19. A composition comprising the amyloid-β protein assembly of claim 1.

20. A vaccine comprising the amyloid-β protein assembly of any claim 1.

21. An antibody that binds to the amyloid-β protein assembly of claim 1.

22-25. (canceled)

26. A method of treating a cognitive disorder in a subject, the method comprising administering an antibody of claim 21 to the subject.

27. A method of detecting a cognitive disorder in a subject, the method comprising contacting a fluid or tissue taken from the subject with an antibody of claim 21.

28. (canceled)

29. A method of disrupting memory of learned behavior in a non-human mammal, the method comprising administering the amyloid-β protein assembly of claim 1 intracranially.

30-32. (canceled)

33. An animal model comprising a non-human mammal wherein an amyloid-β protein assembly of claim 1 has been administered intracranially.

34-36. (canceled)

37. A method of screening for an agent effective for the treatment of a cognitive disorder, the method comprising:
administering a test agent to a first animal to which a soluble amyloid-β protein assembly of claim 1 has been intracranially administered;
measuring cognitive function of the first animal;
comparing the cognitive function of the first animal to the cognitive function of a second animal to which a soluble amyloid-β protein assembly of claim 1 has been intracranially administered, but no test agent has been administered;
wherein an improvement in the cognitive function of the first animal compared to the cognitive function of the second animal indicates the test agent is an effective agent for the treatment of a cognitive disorder.

38-42. (canceled)

43. A method of detecting a cognitive disorder in a subject, the method comprising detecting in a fluid or tissue taken from the subject soluble amyloid-β protein assemblies of claim 1.

44. (canceled)

45. A method for assaying the effects of soluble oligomers of amyloid β protein on cognitive function, the method comprising:
administering a soluble amyloid-β protein assembly of claim 1 intracranially into an animal;
measuring cognitive function to determine the disruption of cognitive behavior.

46-49. (canceled)

50. A method of isolating soluble amyloid-β protein assemblies, the method comprising more than one detergent-stable oligomer of amyloid-β proteins, the method comprising:
homogenizing neuronal tissue in a lysis buffer;
size fractionating amyloid-β protein assemblies; and
isolating an amyloid-β protein assembly of a desired size.
